(19) United States
(12) Patent Application Publication
(10) Pub. No.: US 2005/0177316 A1
(43) Pub. Date: Aug. 11, 2005

(54) Algorithm for estimating and testing association between a haplotype and quantitative phenotype

(75) Inventors: Naoyuki Kamatani, Tokyo (JP); Toshikazu Ito, Tokyo (JP); Yutaka Kitamura, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Mitsubishi Research Institute, Inc., Tokyo (JP); StaGen. Co., Ltd., Ichikawa-shi (JP)

(21) Appl. No.: 10/944,821

(22) Filed: Sep. 21, 2004

(30) Foreign Application Priority Data
Sep. 30, 2003 (JP) 2003-340598
Sep. 16, 2004 (JP) 2004-270432

(51) Int. Cl. G06F 19/00; G01N 33/48; G01N 33/50

(52) U.S. Cl. 702/20

(57) ABSTRACT

A method of estimating, in addition to haplotype frequencies and diplotype configurations, a means and a standard deviation determining a distribution of a quantitative phenotype by the diplotype on the basis of data on observed genotypes and phenotype data taking a continuous value. The method includes a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration including a predetermined haplotype and a predetermined phenotype, and maximum likelihood estimates and the maximum likelihood (Lₘₐₓ) of haplotype frequencies and penetration rate obtained by maximizing the likelihood under the hypothesis that there is an association between the diplotype configuration including the predetermined haplotype and the phenotype distribution taking a continuous value, and a step b of obtaining the means and the standard deviation determining a distribution of a quantitative phenotype from the maximum likelihood estimates obtained in the step a.

### BACKGROUND OF THE INVENTION

1. Field of the Invention

The present invention relates to a method of estimating a means and a standard deviation determining a distribution of a quantitative phenotype using genotype data and continuous-value phenotype data observed in a predetermined population obtained as a result of a cohort study or a clinical trial. The present invention also relates to a method of testing the association between a haplotype and a quantitative phenotype by using estimated values obtained by the method of estimating a means and a standard deviation determining a distribution of a quantitative phenotype.

2. Background Art

Analysis of polymorphism based on linkage disequilibrium (LD) and haplotype structure is becoming increasingly important. A combination of a plurality of linked alletes existing in one gamete is defined here as a haplotype. Also, non-independence of different alletes is defined as linkage disequilibrium. An allele represents a polymorphism in a one of a pair of chromosomes in terms of molecular biology.

Recent studies analyzing data on multiple linked loci (alleles) in many individuals have clarified the structure of the human genome from the viewpoint of the population genetics. That is, it has been made clear that the human genome has a haplotype block (or LD block) structure. Within a block, LD is strong and the number of major haplotypes is limited. One can extract tag SNPs (single nucleotide polymorphism, single base substituted) from the SNPs within the block and use them for association studies. That is, the haplotype structure is very useful for fine mapping of traits.

Haplotypes are also useful for association of genetic information about humans with various phenotypes. A phenotype is often associated not only with each SNP but also with haplotypes. We can interpret haplotypes as complete data and genotypes (e.g., SNP genotypes) as incomplete data. It is because we can extract all genotype data from haplotype data, while the reverse is not the case.

In fact, we can redefine an allele at a locus as a set of multiple haplotypes that have the allele at the locus. Therefore, it is more general to consider the association between a polymorphism and a phenotype based on a haplotype or a diplotype configuration (a combination of haplotypes) rather than based on allele or genotypes. Many studies recently made have suggested that phenotypes such as diabetes and reaction to drugs are associated with haplotypes or diplotype configurations rather than genotypes such as SNP.

However, a haplotype or a diplotype configuration of a subject cannot easily be observed although instances of experimental direct determination of haplotypes have been reported. Haplotypes are inferred by various algorithms using multilocus genotype data instead of being directly observed. That is, Clark's algorithm, the expectation-maximization (EM) algorithm (see non-patent documents 2 to 6), the PHASE algorithm, the PL-EM algorithm, and so on have been proposed.

In genome-based association analysis based on haplotypes, haplotype frequencies are estimated by one of the above-mentioned algorithms. The haplotype frequencies are then compared based on the estimated values among case and control groups. Zaykin et al. have published an algorithm for analysis of the association between haplotype frequencies and traits based on an regression-based approach even in the presence of diplotype configurations not concentrated with respect to discrete or continuous traits (see non-patent document 7). Fallin et al. have also proposed a method of testing the associations between estimated haplotype frequencies and traits on the basis of case/control sampling design (see non-patent document 8).

However, when phenotypes at the level of individuals are brought into focus, they ought be based on diplotype configurations rather than the haplotypes. This relationship is equivalent to that between genotypes and alletes at a locus. In examining the association between a phenotype and genetic information in some cases, therefore, comparing the proportions of affected subjects among individuals with different diplotype configurations is more effective than comparing the haplotype frequencies among different phenotypes.

To compare the proportions of affected subjects among individuals with different diplotype configurations, the diplotype configuration of each individual is inferred by using one of the above-mentioned algorithms and all the individuals are classified according to the presence or absence of a haplotype or a diplotype configuration. The affected and non-affected subjects in populations classified in this manner are further classified and a test as to the independence is thereafter carried out.

However, the problem is that, at least in some individuals, diplotype configurations are not unequivocally determined (only ambiguous diplotype configurations are obtained). The degree of type I error due to classification of individuals according to inferred haplotype information used instead of real haplotype information cannot be distinctly ascertained.

[Non-patent document 1]

Clark A G, Weiss K M, Nickerson D A et al (1998) Haplotype structure and population genetic inferences from nucleotide-sequence variation in human lipoprotein lipase, Am. J. Hum. Genet. 63: 595-612

[Non-patent document 2]

Excoffier L, Slatkin M (1995) Maximum-likelihood estimation of molecular haplotype frequencies in a diploid population, Mol. Biol. Evol. 12: 921-927

[Non-patent document 3]

Hawley ME, Kidd K K (1995) HAPLO: a program using the EM algorithm to estimate the frequencies of multi-site haplotypes, J. Hered. 86: 409-411

[Non-patent document 4]

Schneider S, Roessli D, Excoffier L (2000) Arlequin: a software for population genetics data analysis, Ver 2.000, Genetics and Biometry Laboratory, Department of Anthropology, University of Geneva, Geneva

[Non-patent document 5]

Long J C, Williams R C, Urbanek M (1995) An E-M algorithm and testing strategy for multiple locus haplotypes, Am. J. Hum. Genet. 56: 799-810

[Non-patent document 6]

Kitamura Y, Moriguchi M, Kaneko H, Morisaki H, Morisaki T, Toyama K, Kamatani N (2002), Determination of probability distribution of diplotype configuration (diplotype distribution) for each subject from genotypic data using the EM algorithm, Ann. Hum. Genet. 66: 183-193

[Non-patent document 7]

Zaykin DV, Westfall P H, Young S S, Karnoub M A, Wagner M J, Ehm M G (2002), Testing association of statistically inferred haplotypes with discrete and continuous traits in samples of unrelated individuals, Hum. Hered. 53: 79-91

[Non-patent document 8]

Fallin D, Schork N J (2000), Accuracy of haplotype frequency estimation for biallelic loci, via the expectation-maximization algorithm for unphased diploid genotype data, Am. J. Hum. Genet. 67: 947-959

In view of the above-described problems, an object of the present invention is to provide a method of estimating, as well as haplotype frequencies and diplotype configurations, a means and a standard deviation determining a distribution of a quantitative phenotype by the diplotype on the basis of data on observed genotypes and phenotype data taking a continuous value, and a method of testing the association between a haplotype and a quantitative phenotype by using estimates obtained by using the method of estimating a means and a standard deviation determining a distribution of a quantitative phenotype.

### SUMMARY OF THE INVENTION

An algorithm with which the above-described object of the present invention is achieved makes it possible to perform maximum likelihood estimation of haplotype frequencies in a population, a distribution of diplotypes of individuals (a distribution of posterior probabilities of diplotype configurations) and a means and a standard deviation determining a distribution of a quantitative phenotype by using given genotype data and phenotype data taking a continuous value, with no need to determine the diplotype configurations of the individuals without question. The algorithm in accordance with the present invention is used to enable, for example, a test on the association between the existence of a haplotype and a quantitative phenotype using genotype data and continuous-value phenotype data obtained from a cohort study or a clinical trial. The inventors of the present invention have examined the effectiveness of this algorithm by using both simulations and real data, found that this algorithm is advantageously effective in analysis of genotype data and continuous-value phenotype data obtained by a cohort study or a clinical trial, and achieved the present invention.

That is, the present invention includes the following aspects.

(1) A method of estimating a means and a standard deviation determining a distribution of a quantitative phenotype, the method comprising:

a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration including a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration including the predetermined haplotype and the phenotype data distribution taking a continuous value; and

a step b of obtaining the means and the standard deviation determining a distribution of a qualitative phenotype from the maximum likelihood estimates obtained in the step a.

(2) The method of estimating a means and a standard deviation determining a distribution of a quantitative phenotype described in (1), wherein, in the step a, the maximum likelihood (Lₘₐₓ) is obtained by maximizing the following expression (I):
over Θ, (I)
\vec{μ}
(which is
\vec{μ}=(μ₁, μ₂, . . . , μ_{L}_{²})
and which is a means of distributions of all possible diplotype configurations) and σ (standard deviation), and the maximum likelihood (L₀ₘₐₓ) is obtained by maximizing the following expression (II):

$$L \text{(} Θ , \vec{\overline{μ}} , σ \text{)} ∝ {\prod }_{i = 1}^{N} {\sum }_{{a}_{k} ∈ {A}_{i}} P ( {d}_{i} = {a}_{k} \vert Θ ) f \text{(} {ψ}_{i} = {ω}_{i} \vert {d}_{i} = {a}_{k} , \vec{\overline{μ}} , σ \text{)} \\ over Θ , \vec{\overline{μ}} \text{(} which is \vec{\overline{μ}} = ( {μ}_{1} , {μ}_{1} , … , {μ}_{1} ) \qquad ( II )$$

and which is a means constant with respect to all possible diplotype configurations) and σ (standard deviation)
(wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢωᵢ|dᵢ=aₖ, \vec{μ}, σ)
in the above expression (I) denotes a probability density function for development of a quantitative phenotype x under
di∈D+
where D+ is a set of diplotype configurations including an element in a set of haplotypes related to the predetermined phenotype, and dᵢ is a diplotype configuration of the ith individual in N individuals;

$$f \text{(} {ψ}_{i} = {ω}_{i} \vert {d}_{i} = {a}_{k} , \vec{\overline{μ}} , σ \text{)}$$

in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

(3) The method of estimating a means and a standard deviation determining a distribution of a quantitative phenotype described in (1), wherein genotype data and phenotype data observed in a predetermined population and obtained as a result of a cohort study or a clinical trial are used.

(4) The method estimating a means and a standard deviation determining a distribution of a quantitative phenotype described in (1), wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.

(5) A method of testing the association between a haplotype and a quantitative phenotype, the method comprising:

a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration having a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration including the predetermined haplotype and the phenotype data distribution taking a continuous value; and

a step b of obtaining a likelihood ratio from the maximum likelihood (L₀ₘₐₓ) and the maximum likelihood (Lₘₐₓ) obtained in the step a, and testing, with respect to x² distribution, the hypothesis that there is an association between the diplotype configuration including the predetermined haplotype and the predetermined quantitative phenotype.

(6) The method of testing an association described in (5), wherein, in the step a, the maximum likelihood (Lₘₐₓ) is obtained by maximizing the following expression (I):

$$L ( Θ , \vec{μ} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , \vec{μ} , σ ) \\ over Θ , \vec{μ} \text{(} which is \vec{μ} = ( {μ}_{1} , {μ}_{2} , … , {μ}_{{L}^{2}} ) \qquad ( I )$$

and which is a means of distributions of all possible diplotype configurations) and σ (standard deviation), and the maximum likelihood (L₀ₘₐₓ) is obtained by maximizing the following expression (II):

$$L \text{(} Θ , \vec{\overline{μ}} , σ \text{)} ∝ {\prod }_{i = 1}^{N} {\sum }_{{a}_{k} ∈ {A}_{i}} P ( {d}_{i} = {a}_{k} \vert Θ ) f \text{(} {ψ}_{i} = {ω}_{i} \vert {d}_{i} = {a}_{k} , \vec{\overline{μ}} , σ \text{)} \\ over Θ , \vec{\overline{μ}} \text{(} which is \vec{\overline{μ}} = ( {μ}_{1} , {μ}_{1} , … , {μ}_{1} ) \qquad ( II )$$

and which is a means constant with respect to all possible diplotype configurations) and σ (standard deviation)
(wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢωᵢ|dᵢ=aₖ, \vec{μ}, σ)
in the above expression (I) denotes a probability density function for development of a quantitative phenotype x under
dᵢ∈D₊
where D₊ is a set of diplotype configurations including an element in a set of haplotypes related to the predetermined phenotype, and dᵢ is a diplotype configuration of the ith individual in N individuals;

$$f \text{(} {ψ}_{i} = {ω}_{i} \vert {d}_{i} = {a}_{k} , \vec{\overline{μ}} , σ \text{)}$$

in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

(7) The method of testing an association described in (5), wherein, in the step b, −2log(Lₘₐₓ/L₀ₘₐₓ) (where “log” denotes natural logarithm) is obtained as a statistic, and wherein, in a case where there is no association between the diplotype configuration including the predetermined haplotype and the quantitative phenotype, and where the statistic therefore follows asymptotically x² distribution with 1 degree of freedom, it is determined that no association can be said to exist between the diplotype configuration including the predetermined haplotype and the predetermined quantitative phenotype, when the statistic does not exceed the limit value x² (which is a value in x² distribution with 1 degree of freedom at which a cumulative distribution function becomes 1−α (where α is a level of significance of the test)), and it is determined that there is an association between the predetermined haplotype and the predetermined quantitative phenotype, when the statistic exceeds the limit value x².

(8) The method of testing an association described in (5), wherein genotype data and phenotype data taking a continuous value observed in a predetermined population and obtained as a result of a cohort study or a clinical trial are used.

(9) The method of testing an association described in (5), wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.

The present invention also provides a program for enabling a computer to execute the steps in any of the above-described steps (1) to (9).

### BRIEF DESCRIPTION OF THE DRAWINGS

FIG. 1 comprises histograms relating to distributions of a test statistic −2log(L₀ₘₐₓ/Lₘₐₓ) obtained by simulation using a 4-locus haplotype distribution.

FIG. 2 is a characteristic diagram showing the power of a test with respect to γ=(μ₁-μ₂)σ.

FIG. 3 comprises characteristic diagrams showing distributions of test data obtained by a study on relation between the CAPN10 gene and diabetes.

### DETAILED DESCRIPTION OF THE PREFERRED EMBODIMENTS

The present invention will be described below in detail.

The method of estimating a means and a standard deviation determining a distribution of a quantitative phenotype and the method of testing the association between a diplotype and a quantitative phenotype in accordance with the present invention are implemented by means of an algorithm described below. In the algorithm used in the present invention (hereinafter referred to as “the present algorithm”), the association between the existence of a quantitative phenotype and the existence of a predetermined haplotype is tested by using genotype data and continuous-value phenotype data on individuals obtained as a result of a cohort study or a clinical trial, and a means and a standard deviation determining a distribution of the quantitative phenotype on the basis of the haplotype are estimated. The present algorithm is formed on the basis of an expectation-maximization (EM) algorithm.

The present algorithm enables estimation of a means and a standard deviation determining a distribution of a quantitative phenotype varying among individuals having a predetermined haplotype and individuals not having the predetermined haplotype, as well as the haplotype frequency in the population. Therefore, the present algorithm enables maximum-likelihood estimation of a relative risk.

In the present invention, “continuous-value phenotype data” represents data on a phenotype taking a continuous value, e.g., clinical examination data such as the blood pressure value, the concentration of a drug in blood, a dose, the amount of expression of a gene in a DNA microarray, or the amount of expression of a protein. This phenotype represents a phenotype in a quantitative trait locus (QTL). Phenotype data taking a continuous value will be hereinafter referred to as “QTL phenotype”. “A means and a standard deviation determining a distribution of a quantitative phenotype” are parameters relating to a distribution of phenotype data taking a continuous value. “A means and a standard deviation determining a distribution of a quantitative phenotype” are, in other words, “the ranges of values for a QTL phenotype”.

More specifically, in the present algorithm, the maximum likelihood (L₀ₘₐₓ) under a hypothesis that there is no haplotype associated with a continuous-value phenotype (the null hypothesis) and the maximum likelihood (Lₘₐₓ) under a hypothesis that there is haplotype associated with a continuous-value phenotype (the alternative hypothesis) are first calculated. Next, in the present algorithm, a statistic, e.g., −2log(L₀ₘₐₓ/Lₘₐₓ) (hereinafter referred to simply as “statistic”) is computed. The association between the continuous-value phenotype and the haplotype is tested on the basis of this statistic.

The present algorithm can be applied to a method of estimating a means and a standard deviation determining a distribution of a quantitative phenotype in specimens from genotype information on the specimens. That is, with the present algorithm, a means and a standard deviation determining a distribution of a predetermined quantitative phenotype in specimens can be estimated from genotype information on the specimens. Therefore, the present algorithm is particularly suitable for analysis of the association between a genetic factor and a QTL phenotype in individuals taking a continuous value, e.g., clinical examination data such as the blood pressure value, the concentration of a drug in blood, a dose, the amount of expression of a gene in a DNA microarray, or the amount of expression of a protein.

The present algorithm can be executed on a computer by being implemented in the computer software QTLhaplo. The computer has a central processing unit (CPU) for overall control on the operation, an input apparatus such as a keyboard and a mouse capable of inputting an instruction for executing a program, etc., a display apparatus such as a display device, a memory in which temporary information, a program and the like are stored, and a storage such as a hard disk on which various sorts of data, a program and the like are stored. The computer may be connected to an external database, another computer, and the like via a communication network such as the Internet.

For execution of the present algorithm on the computer, the computer software QTLhaplo is installed. The computer can execute the present algorithm in accordance with the computer program QTLhaplo under the control of the CPU. Genotype data and continuous-value phenotype data can also be obtained via a communication network, e.g., the Internet or the like.

That is, the present algorithm enables the computer to function as an input means for obtaining genotype data and continuous-value phenotype data and as a control means (computation means) for obtaining maximum-likelihood estimates and maximum likelihoods (Lₘₐₓ) of the means and the standard deviation of a haplotype frequency and a distribution of a quantitative phenotype by using the genotype data and continuous-value phenotype data obtained by the input means.

The present algorithm also enables the computer to function as the control means to obtain the likelihood ratio from the maximum likelihood (L₀ₘₐₓ) and the maximum likelihood (Lₘₐₓ) and test, with reference to x² distribution, a hypothesis that there is an association between a predetermined diplotype configuration and a predetermined quantitative phenotype.

The above-mentioned genotype data comprises information denoting the position of a polymorphism obtained as a result of execution of so-called SNP typing or the like on a certain individual, and information denoting the kind of the polymorphism. The genotype data may be made anonymous by removing information identifying the individual.

The continuous-value phenotype data comprises data on a certain individual denoting a predetermined numeric value in the continuous value of a quantitative phenotype, or data denoting being within or out of a predetermined range of the continuous value of a quantitative phenotype.

A diplotype configuration is defined as an ordered combination of two haplotype copies. Let a₁, a₂, . . . , a_{L2} be possible diplotype configurations. The probability that ith individual has the diplotype configuration aₖ is P(dᵢ=aₖ|Θ)=Θ₁Θₘ. In this expression, dᵢ is a random variable representing a diplotype configuration for the ith individual, and 1 and m denote the orders of the haplotypes constituting aₖ. This means that Hardy-Weinberg's equilibrium at the haplotype level is assumed.

The ith individual develops a QTL phenotype φᵢ that follows a probability density function. The QTL phenotype is assumed to follow a normal distribution with a means dependent on dᵢ and with a fixed standard deviation independent of dᵢ. A set of experimental results is expressed as (Θ, D, Ψ), D=(d₁, . . . , d_{N}) denotes the vectors of the diplotype configurations in the individuals i, and Ψ(Ψ₁, . . . , Ψ_{N}) denotes the vectors of QTL phenotypes in the individuals i. The means μ is assumed to be dependent on whether or not di contains the haplotype h_{b} relating to a phenotype differing in influence from the others. D+denotes a set of diplotype configurations that contain the haplotype h_{b}. Only two normal distributions are then defined for a QTL phenotype dependent on the diplotype configuration. One of them is N(μ₁, σ) for the distribution with
dᵢ∈D₊
and the other is N(μ₂, σ) for those with
dᵢ∉D₊

Let fμ₁(x) denote the probability density function for development of a QTL phenotype x in the ith individual when dᵢ ∈D₊, and let fμ₂(x) denote the probability density function for development of the QTL phenotype x in the ith individual when dᵢ∉D₊.

If φi denotes the QTL phenotype of the ith individual,
ƒ(ψ=x|dᵢ∈D₊)=ƒ_{μ1}(x)
and
ƒ(ψ=x|dᵢ∉D₊)=ƒ_{μ2}(x)
Note that Θ is independent of each of fμ₁(x) and fμ₂(x), and that φᵢ is independent of Θ conditional on dᵢ.

In the present algorithm, it is theoretically possible to assume distribution functions for quantitative phenotypes specified by using means and standard deviations with respect to all the diplotype configurations. However, it is not realistic to assign the distribution functions to all the diplotype configurations. In the present algorithm, therefore, only two distribution functions fμ₁(x) and fμ₂(x) are assumed, as described above. The haplotype h_{b} associated with a phenotype to be tested is not limited to only one haplotype. It may be defined as a subset H+ of the haplotypes. That is, if Hₐₗₗ is the set of all the haplotypes, H₊ is a subset of Hₐₗₗ the elements of which include the haplotype or haplotypes that presence of which has a different effect than the others contained in the diplotype configuration. H₊ typically contains only one haplotype, but may contain a plurality of haplotypes as elements. H+ is a set of haplotypes to be tested in the present algorithm. If H₊ is defined as a set of all the haplotypes with a particular allele at a particular locus, the testing in this embodiment is equivalent to testing the association between an allele (rather than a haplotype) and a quantitative phenotype.

The subset of Hₐₗₗ is not limited to H₊. H_{I} described below may be defined. H_{I} is defined as a subset of Hₐₗₗ in such a manner that loci for giving information for discrimination between individual haplotypes and loci having redundant information depending on a combination of the loci having the discrimination information are identified on the basis of a haplotype distribution inferred by the EM algorithm, and the identified loci are masked.

This masking means concealing the information at particular one or more of the plurality of loci constituting the haplotypes by assuming that any polymorphism can apply to the particular locus or loci. Thus, the concept of an incomplete haplotype having its portion concealed by masking is expressed as a set H_{I} containing a plurality of haplotypes as elements. From the definition of H_{I},
H_{I} [symbol] Hₐₗₗ
is apparent. Further, if an incomplete haplotype is constructed by using information on only one locus and by masking the other loci, only SNP information on the used locus is used. This incomplete haplotype is therefore equivalent to SNP. If haplotypes formed in this manner are defined as a set H_{SNP}, H_{SNP} is a particular case of H_{I}, and
H_{I} [symbol] H_{SNP} [symbol] Hₐₗₗ

The rationality of using incomplete haplotypes H_{I} as objects to be tested in place of H+ is as described below.

1) In a case where a gene polymorphism is expressed by haplotypes, the cause of the polymorphism comprises base substitution and recombination. In a case where a haplotype in a certain region is associated with a cause locus associated with a certain quantitative phenotype, a plurality of haplotypes may be associated with the cause locus. This association is due to a mutation or recombination caused after the occurrence of the cause locus. If incomplete haplotypes are constructed, a mutation can be expressed as masking at a particular locus and recombination can be expressed as masking at consecutive loci.

2) When incomplete haplotypes are constructed by using SNP information on L loci, the loci to be masked are changed from 0 to L-1 to enable haplotypes from those using all the information items to SNP to be included in objects to be tested by the present algorithm.

If all incomplete haplotypes H_{I} are constructed by using SNP information on the L loci, three information items consisting of two alleles and a masking operation are used with respect to each locus and, to put is simply, there is a need to consider 3^{L}-1 combinations. The number of combinations is excessively large even in comparison with the number of combinations 2L in haplotype estimation. However, the number of haplotypes is ordinarily smaller than 10 in a region where linkage disequilibrium is strong, and it is not necessary to simply construct all the possible combinations. Therefore, incomplete haplotypes H_{I} may be constructed in accordance with an algorithm having the following steps 1 to 3.

1. Haplotype estimation based on the EM algorithm is performed.

2. From a haplotype distribution thereby estimated, loci for giving information for discrimination between individual haplotypes are extracted. From the loci thereby extracted, those having redundant information due to combinations are removed to determine haplotype tags SNP (hereinafter referred to as htSNP).

3. To the htSNP loci, three information items consisting of two alleles and a masking operation are successively applied to construct incomplete haplotypes H_{I}. There is a possibility of identical haplotypes H_{I} being constructed even by different masking methods. A redundant haplotype constructed in such a case is also removed.

Description will be made with respect to a case where H+ is provided as an object to be tested. However, the above-described incomplete haplotypes H_{I} may alternatively be provided as an object to be tested.

<Likelihood Function>

The observed data used in the present algorithm are genotype data and QTL phenotype data on individuals. Let G_{obs}=(g₁, g₂, . . . , g_{N}) and Ψ_{obs}=(w₁, w₂, . . . , w_{N}) denote the vectors of the observed genotype and phenotypes data, respectively, where gᵢ and wᵢ denote the observed genotypes and QTL phenotype of the ith individual. The means of the distributions for all possible diplotype configurations is defined as follows:
\vec{μ}=(μ₁, μ₂, . . . , μ_{L}_{²})
Then the likelihood function is

$$L ( Θ , \vec{μ} , σ ) ∝ {\prod }_{i = 1}^{N} {\sum }_{{a}_{k} ∈ {A}_{i}} P ( {d}_{i} = {a}_{k} \vert Θ , \vec{μ} , σ ) f ( {ψ}_{i} = {ω}_{i} \vert {d}_{i} = {a}_{k} , \vec{μ} , σ )$$

where Aᵢ denotes the set of aₖ for the ith individual that is consistent with gᵢ. Since dᵢ is independent of
\vec{μ}
and σ, and since φ₁ is independent of Θ conditional on dᵢ,

$$L ( Θ , \vec{μ} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , \vec{μ} , σ ) \qquad ( I )$$

where Aᵢ denotes the set of diplotype configurations for the ith individual that are consistent with gᵢ. The ith individual develops QTL phenotype x in accordance with the following probability density function:

$$f ( {ψ}_{i} = x \vert {d}_{i} = {a}_{k} , \vec{μ} , σ ) = \{ \begin{matrix}( \frac{1}{\sqrt{2 πσ}} ) ⅇ - \frac{{( x - {μ}_{1} )}^{2}}{2 {σ}^{2}} = {f}_{μ 1} ( x ) \\ ( \frac{1}{\sqrt{2 πσ}} ) ⅇ - \frac{{( x - {μ}_{2} )}^{2}}{2 {σ}^{2}} = {f}_{μ 2} ( x )\end{matrix}$$

Under the null hypothesis that the distribution of a QTL phenotype is independent of the diplotype configuration, the likelihood function is

$$L \text{(} Θ , \vec{\overline{μ}} , σ \text{)} ∝ {\prod }_{i = 1}^{N} {\sum }_{{a}_{k} ∈ {A}_{i}} P ( {d}_{i} = {a}_{k} \vert Θ ) f \text{(} {ψ}_{i} = {ω}_{i} \vert {d}_{i} = {a}_{k} , \vec{\overline{μ}} , σ \text{)} \qquad ( II )$$

Under the null hypothesis, the means of the distribution of a QTL phenotype is invariable regardless of the diplotype configuration, and is expressed by

$$\vec{\overline{μ}} = ( {μ}_{1} , {μ}_{1} , … , {μ}_{1} )$$

Aᵢ is the set of diplotype configurations for the ith individual that are consistent with gᵢ.
<EM Algorithm>

In the present algorithm, expression (I) is maximized over Θ,

\vec{μ}

and σ, and the maximum likelihood thereby obtained is denoted as Lₘₐₓ. In the present algorithm, expression (II) is also maximized over Θ,

$$\vec{\overline{μ}}$$

and σ, and the maximum likelihood thereby obtained is denoted as L₀ₘₐₓ. In the present algorithm, the likelihood ratio L₀ₘₐₓ/Lₘₐₓ is used to test the association between the existence of the haplotypes and the QTL phenotypes.

In the maximization for Lₘₐₓ, the parameters to be estimated are Θ=(θ₁, θ₂, . . . , θ_{L}),

\vec{μ}

and σ, while in the maximization for L₀ₘₐₓ, the parameters to be estimated are Θ=(θ₁, θ₂, . . . , θₗ)

$$\vec{\overline{μ}}$$

and σ. Under the null hypothesis, −2log(L₀ₘₐₓ/Lₘₐₓ) is expected to follow the x² distribution with 1 degree of freedom.

If the complete data for d₁, d₂, . . . , d_{N} and φ₁, φ₂, . . . , φ_{N} were available, the maximum likelihood estimates of θ₁, θ₂, . . . , θ_{L},

\vec{μ}

and σ, would be obtained as

$$\begin{matrix}{{θ}^{⋒}}_{j} = {n}_{j} / ( 2 N ) \\ {{μ}^{⋒}}_{1} = {\sum }_{{d}_{i} ∈ {D}_{+}} {ψ}_{i} / {N}_{+} \\ {{μ}^{⋒}}_{2} = {\sum }_{{d}_{i} ∉ {D}_{+}} {ψ}_{i} / {N}_{-} \\ {σ}^{⋒} = \sqrt{( ( {\sum }_{{d}_{i} ∈ {D}_{+}} {( {ψ}_{i} - {μ}_{1} )}^{2} + {\sum }_{{d}_{i} ∉ {D}_{+}} {( {ψ}_{i} - {μ}_{2} )}^{2} ) / N}\end{matrix}$$

where j=1, 2, . . . , L is the haplotype number, nⱼ is the number of copies of the jth haplotype in the individuals, N₊ denotes the number of individuals having the haplotype h_{b}, and N₋ denotes the number of individuals not having the haplotype h_{b}. However, the complete data are not available, and only genotypes and QTL phenotypes of the individuals can be observed. Therefore, the expected values of

$$\begin{matrix} {n}_{j} / ( 2 N ) \\ {\sum }_{{d}_{i} ∈ {D}_{+}} {ψ}_{i} / {N}_{+} \\ {\sum }_{{d}_{i} ∉ {D}_{+}} {ψ}_{i} / {N}_{-} \\ \sqrt{( {\sum }_{{d}_{i} ∈ {D}_{+}} {( {ψ}_{i} - {μ}_{1} )}^{2} + {\sum }_{{d}_{i} ∉ {D}_{+}} {( {ψ}_{i} - {μ}_{2} )}^{2} ) / N}\end{matrix}$$

are substituted for the real values in the following EM algorithm (steps 1 to 9).
Step 1

For n=0, initial values are given to

$${Θ}^{( n )} = ( {θ}_{1}^{( n )} , {θ}_{2}^{( n )} , … , {θ}_{L}^{( n )} ) where$$

$${\sum }_{j = 1}^{L} {θ}_{j}^{( n )} = 1 and$$

$${θ}_{j}^{( n )} > 0$$

Step 2

For n=0, initial values are given to
\vec{n μ}=(μ₁⁽ⁿ⁾, μ₂⁽ⁿ⁾)
Step 3

For n=0, σ⁽ⁿ⁾ is given as an initial value to the standard deviation. It is assumed that σ takes the same value regardless of whether or not the individual has the haplotype h_{b}.

Step 4

For all the individuals i and for permutated diplotype configurations aₖ consistent with

$${g}_{i} , \\ P ( {d}_{i} = {a}_{k} \vert {ψ}_{i} = {ω}_{i} , {Θ}^{( n )} , {\vec{μ}}^{( n )} , {σ}^{( n )} ) = \\ P ( {d}_{i} = {a}_{k} \vert {Θ}^{( n )} , {\vec{μ}}^{( n )} , {σ}^{( n )} ) f ( {ψ}_{i} = {ω}_{i} \vert {d}_{i} = {a}_{k} , {Θ}^{( n )} , {\vec{μ}}^{( n )} , {σ}^{( n )} 〉 / \\ {\sum }_{{a}_{m} ∈ {A}_{i}} P ( {d}_{i} = {a}_{m} \vert {Θ}^{( n )} , {\vec{μ}}^{( n )} , {σ}^{( n )} ) f ( {ψ}_{i} = {ω}_{i} \vert {d}_{i} = {a}_{m} , {Θ}^{( n )} , {\vec{μ}}^{( n )} , {σ}^{( n )} 〉 \qquad ( III )$$

is calculated. In this equation, Aᵢ denotes the set of diplotype configurations aₘ consistent with gᵢ in the individuals i. Note that the calculation is performed only with respect to only aₖ consistent with gᵢ. Further, since dᵢ is independent of
\vec{n μ}
and σ⁽ⁿ⁾, and since φ₁ is independent of Θ⁽ⁿ⁾ conditional on dᵢ, equation (III) shown above becomes

$$P ( {d}_{i} = {a}_{k} \vert {ψ}_{i} = {ω}_{i} , {Θ}^{( n )} , {\vec{μ}}^{( n )} , {σ}^{( n )} ) = \\ P ( {d}_{i} = {a}_{k} \vert {Θ}^{( n )} ) f ( {ψ}_{i} = {ω}_{i} \vert {d}_{i} = {a}_{k} , {\vec{μ}}^{( n )} , {σ}^{( n )} 〉 / \\ {\sum }_{{a}_{m} ∈ {A}_{i}} P ( {d}_{i} = {a}_{m} \vert {Θ}^{( n )} ) f ( {ψ}_{i} = {ω}_{i} \vert {d}_{i} = {a}_{m} , {\vec{μ}}^{( n )} , {σ}^{( n )} 〉 \qquad ( IV )$$

Step 5

Since nⱼ, the number of the jth haplotype copies in the N individuals is a random variable, the expected number of the jth haplotype copies can be defined as

$$E [ {n}_{j} \vert {Ψ}_{obs} , {G}_{obs} , {Θ}^{( n )} , {\vec{μ}}^{( n )} , {σ}^{( n )} ] = \\ {\sum }_{i = 1}^{N} {\sum }_{{a}_{k} ∈ {A}_{i}} {g}_{i} ( {a}_{k} ) P ( {d}_{i} = {a}_{k} \vert {Ψ}_{obs} , {G}_{obs} , {Θ}^{( n )} , {\vec{μ}}^{( n )} , {σ}^{( n )} )$$

where gⱼ(aₖ) denotes the number of the jth haplotype copies in aₖ, and Aᵢ, again, denotes the set of diplotype configurations consistent with gᵢ in the individuals i. Note that gⱼ(aₖ) is either 0, 1 or 2. The expected values are calculated for all j.
Step 6
Σdᵢ∈D₊ψᵢ/N₊
Σdᵢ∉D₊ψᵢ/N₋
and
{square root over ((Σdᵢ∈D₊(ψᵢ−μ₁)²+Σdᵢ∉D₊(ψᵢ−μ₂)²)/N)}
are random variables and, therefore, expected values thereof can be defined.

First, the expected value of
Σdᵢ∈D₊ψᵢ/N₊
is defined by the following equations:

$$E [ {\sum }_{{d}_{i} ∈ {D}_{+}} {ψ}_{i} / {N}_{+} \vert {Ψ}_{obs} , {G}_{obs} , {Θ}^{( n )} , {\vec{μ}}^{( n )} , {σ}^{( n )} ] = \frac{{\sum }_{i = 1}^{N} {ψ}_{i} ( {u}_{b} / {u}_{0} )}{{\sum }_{i = 1}^{N} ( {u}_{b} / {u}_{0} )}$$

$${u}_{b} = {\sum }_{{d}_{i} ∈ {D}_{+}} P ( {d}_{i} = {a}_{k} \vert {ψ}_{i} = {ω}_{i} , {Θ}^{( n )} , {\vec{μ}}_{1}^{( n )} , {σ}^{( n )} ) f ( {ψ}_{i} \vert {d}_{i} = {a}_{k} , {μ}_{1}^{( n )} , {σ}^{( n )} )$$

$${u}_{0} = {\sum }_{{a}_{k} ∈ {A}_{i}} P ( {d}_{i} = {a}_{k} \vert {ψ}_{i} = {ω}_{i} , {Θ}^{( n )} , {\vec{μ}}_{1}^{( n )} , {σ}^{( n )} ) f ( {ψ}_{i} \vert {d}_{i} = {a}_{k} , {μ}_{1}^{( n )} , {σ}^{( n )} )$$

The numerator and the denominator in the above factional expression are respectively weighted by the proportion of the set containing the haplotype related to a phenotype to the set of perinutated diplotype configurations of the individuals consistent with gᵢ, i.e., u_{b}/u₀.

Next, the expected value of
Σdᵢ∈D₊ψᵢ/N₋
is defined by the following equations:

$$E [ {\sum }_{{d}_{i} ∉ {D}_{+}} {ψ}_{i} / {N}_{-} \vert {Ψ}_{obs} , {G}_{obs} , {Θ}^{( n )} , {\vec{μ}}^{( n )} , {σ}^{( n )} ] = \frac{{\sum }_{i = 1}^{N} {ψ}_{i} ( {v}_{b} / {v}_{0} )}{{\sum }_{i = 1}^{N} ( {v}_{b} / {v}_{0} )}$$

$${v}_{b} = {\sum }_{{a}_{k} ∉ {D}_{+}} P ( {d}_{i} = {a}_{k} \vert {ψ}_{i} = {ω}_{i} , {Θ}^{( n )} , {\vec{μ}}_{2}^{( n )} , {σ}^{( n )} ) f ( {ψ}_{i} \vert {d}_{i} = {a}_{k} , {μ}_{2}^{( n )} , {σ}^{( n )} )$$

$${v}_{0} = {\sum }_{{a}_{k} ∈ {A}_{i}} P ( {d}_{i} = {a}_{k} \vert {ψ}_{i} = {ω}_{i} , {Θ}^{( n )} , {\vec{μ}}_{2}^{( n )} , {σ}^{( n )} ) f ( {ψ}_{i} \vert {d}_{i} = {a}_{k} , {μ}_{2}^{( n )} , {σ}^{( n )} )$$

The numerator and the denominator in this case are respectively weighted by the proportion of the set not containing the haplotype related to the phenotype to the set of permutated diplotype configurations of the individuals i consistent with gᵢ, i.e., v_{b}/v₀.

Further, the expected value of
{square root over ((Σdᵢ∈D₊(ψᵢ−μ₁)²+Σdᵢ∉D₊(ψᵢ−μ₂)²)/N)}
is defined by the following equations:

$$\begin{matrix}E [ \sqrt{( {\sum }_{{d}_{i} ∈ {D}_{+}} {( {ψ}_{i} - {μ}_{1} )}^{2} + {\sum }_{{d}_{i} ∈ {D}_{+}} {( {ψ}_{i} - {μ}_{2} )}^{2} ) / N} \vert {Ψ}_{obs} , \\ {G}_{obs} , {Θ}^{( n )} , {\vec{μ}}^{( n )} , {σ}^{( n )} ] = \{ \frac{1}{n} {\sum }_{i = 1}^{N} {( {ψ}_{i} - {μ}_{1} )}^{2} {\sum }_{i = 1}^{N} ( {u}_{b} / {u}_{0} ) + \\ { \frac{1}{n} {\sum }_{i = 1}^{N} {( {ψ}_{i} - {μ}_{2} )}^{2} {\sum }_{i = 1}^{N} ( {v}_{b} / {v}_{0} ) \}}^{\frac{1}{2}}\end{matrix}$$

In this equation, σ is weighted by the proportion of the set containing the haplotype related to the phenotype to the set of permutated diplotype configurations of the individuals i consistent with gᵢ (i.e., u_{b}/u₀) and the proportion of the set not containing the haplotype related to the phenotype to the set of permutated diplotype configurations of the individuals i consistent with gᵢ (i.e., v_{b}/v₀). Also, n is given by

$${\sum }_{i = 1}^{N} ( {u}_{b} / {u}_{0} ) + {\sum }_{i = 1}^{N} ( {v}_{b} / {v}_{0} )$$

Step 7

To perform calculation in the next step, Θ is updated by using the results of calculation in step 5, as shown below.

$${θ}_{j}^{( n + 1 )} = E ⌊ {n}_{j} \vert {Ψ}_{obs} , {G}_{obs} , {Θ}^{( n )} , {\vec{μ}}^{( n )} , {σ}^{( n )} ⌋ / ( 2 N )$$

To perform calculation in the next step,

\vec{μ}

and σ are updated by using the results of calculation in step 6, as shown below.

$$\begin{matrix}{μ}_{1}^{( n + 1 )} = E [ {\sum }_{{d}_{i} ∈ {D}_{+}} {ψ}_{i} / {N}_{+} \vert {Ψ}_{obs} , {G}_{obs} , {Θ}^{( n )} , {\vec{μ}}^{( n )} , {σ}^{( n )} ] \\ {μ}_{2}^{( n + 1 )} = E [ {\sum }_{{d}_{i} ∈ {D}_{+}} {ψ}_{i} / {N}_{-} \vert {Ψ}_{obs} , {G}_{obs} , {Θ}^{( n )} , {\vec{μ}}^{( n )} , {σ}^{( n )} ] \\ {σ}^{( n + 1 )} = E [ \sqrt{( {\sum }_{{d}_{i} ∈ {D}_{+}} {( {ψ}_{i} - {μ}_{1} )}^{2} + {\sum }_{{d}_{i} ∈ {D}_{+}} {( {ψ}_{i} - {μ}_{2} )}^{2} ) / N} \vert {Ψ}_{obs} , \\ {G}_{obs} , {Θ}^{( n )} , {\vec{μ}}^{( n )} , {σ}^{( n )} ]\end{matrix}$$

Step 8

The steps 4 through 7 are repeated until the values converge. The values when converged are considered as the maximum likelihood estimates:

Θ=(\hat{θ}₁, \hat{θ}₂, . . . , \hat{θ}_{L})

\hat{μ}₁

\hat{μ}₂

and

\hat{σ}

Step 9

To avoid convergence to a local maximum, convergence calculation is repeatedly performed with respect to different sets of initial values:

θⱼ⁽⁰⁾=(j=1,2, . . . ,L)

μ₁⁽⁰⁾

μ₂⁽⁰⁾

and

σ⁽⁰⁾, and the values maximizing the likelihood function are obtained. Here,

$$P ( {Ψ}_{obs} , {G}_{obs} \vert {Θ}^{⋒} , {\vec{μ}}^{⋒} , {σ}^{⋒} )$$

is the maximum likelihood Lₘₐₓ for the alternative hypothesis. If the steps 4 through 7 are repeated on condition that

$$\vec{\overline{μ}} = ( {μ}_{1} , {μ}_{1} , … , {μ}_{1} )$$

the maximum likelihood L₀ₘₐₓ for the null hypothesis is obtained. Under the null hypothesis, the statistic −2log(L₀ₘₐₓ/Lₘₐₓ) is expected to follow x² distribution with 1 degree of freedom.

The above-described EM algorithm and the algorithm for estimating a means and a standard deviation determining a distribution of a quantitative phenotype (present algorithm) can be incorporated in a piece of computer software for example. If the present algorithm is incorporated in a piece of computer software, all the calculation operations can be performed in a computer.

A computer in which a piece of software incorporating the present algorithm is installed may be connected to an external network via a communication network to obtain, for example, through the communication network, genotype data and continuous-value phenotype data on individuals obtained as a result of a cohort study or a clinical trial. Also, the computer can output through the communication network a means and a standard deviation estimate determining a distribution of a quantitative phenotype, estimated by the present algorithm.

<Simulation>

Accuracy of Estimation of Distribution Parameters

The estimation accuracy of the present algorithm was examined by a simulation. The simulation was made by a method of preparing samples by determining genotypes and phenotypes of N individuals from a population with assumed haplotype frequencies and performing estimation and testing on the obtained samples. As haplotype frequencies Θ of the population, haplotype frequencies related to SAA (serum amyloid A) gene, obtained by our study in the past, were used without assuming a population genetics model [Moriguchi et al. (2001) A novel single-nucleotide polymorphism at the 5′-flanking region of SAAI associated with risk of type AA amyloidosis secondary to rheumatoid arthritis. Arthritis Rheum 44:1266-1272]. The haplotype data relating to SAA gene includes 6-locus SNP data. However, linkage disequilibrium at the locus 1 and the locus 4 in the six loci is not so strong. Therefore, a haplotype distribution with respect to the six loci including these loci and a haplotype distribution with respect to the four loci other than these loci were assumed as haplotype distributions in the population. Table 1 shows the haplotype distributions used.

TABLE 1
SAA gene haplotype distributions

| 6-locus haplotypes | | | 4-locus haplotypes | | |
|---|---|---|---|---|---|
| | Relative | QTL | | | QTL |
| | fre- | phenotype | | Relative | phenotype |
| Haplotype | quency | distribution | Haplotype | frequency | distribution |
| ACTGCC | 0.394 | N(μ₂, σ) is | CTCC | 0.391 | N(μ₂, σ) is |
| | | assumed | | | assumed |
| ACCGTC^{a} | 0.214 | N(μ₂, σ) is | GCCT | 0.267 | N(μ₂, σ) is |
| | | assumed | | | assumed |
| ACCGCT | 0.210 | N(μ₂, σ) is | CCTC^{a} | 0.258 | N(μ₂, σ) is |
| | | assumed | | | assumed |
| GCCGTC | 0.036 | N(μ₂, σ) is | CTCT | 0.061 | N(μ₂, σ) is |
| | | assumed | | | assumed |
| GCTGCT | 0.035 | N(μ₂, σ) is | CTTC | 0.013 | N(μ₂, σ) is |
| | | assumed | | | assumed |
| GGCACT | 0.023 | N(μ₂, σ) is | CCCC | 0.007 | N(μ₂, σ) is |
| | | assumed | | | assumed |
| ACTGCT | 0.023 | N(μ₂, σ) is | GCCC | 0.003 | N(μ₂, σ) is |
| | | assumed | | | assumed |
| AGCACT | 0.018 | N(μ₂, σ) is | | | |
| | | assumed | | | |
| GGCGCT | 0.017 | N(μ₂, σ) is | | | |
| | | assumed | | | |
| ACTGTC | 0.013 | N(μ₂, σ) is | | | |
| | | assumed | | | |
| ACCGCC | 0.006 | N(μ₂, σ) is | | | |
| | | assumed | | | |
| ACCATC | 0.006 | N(μ₂, σ) is | | | |
| | | assumed | | | |
| AGCGCC | 0.003 | N(μ₂, σ) is | | | |
| | | assumed | | | |

Ordered two hplotype copies were extracted with respect to each of the N individuals on the basis of the assumed haplotype frequencies to determine genotypes. Further, one of the haplotypes shown in Table 1 was determined as a haplotype related to the QTL phenotype that follows the N (μ₁, σ) distribution. In the case where the haplotype related to the phenotype was held, the QTL phenotype that randomly follows the N (μ₁,σ) distribution was given. In the case where the haplotype related to the phenotype was not held, the QTL phenotype that randomly follows the N (μ₂, σ) distribution was given. Thereafter, the above-described algorithm was applied by removing the phase to estimate the distribution parameters.

The results of test calculation on simulation data using the 4-locus haplotype distribution are shown in the estimation/test result section of Table 2. The values shown in the estimation/test section were obtained under the conditions where μ₁=μ₂=160, σ=5 for the null hypothesis and μ₁≢μ₂σ=5 for alternative hypothesis with respect to different sample sizes N.

TABLE 2
Results of estimation using 4-locus simulation data

| Conditions of | | | | | | | | |
|---|---|---|---|---|---|---|---|---|
| population | | Statistics of samples | | | Estimation/test results | | | |
| (μ₁, μ₂, σ) | N | μ₁ | μ₂ | σ | \hat{μ}₁ | \hat{μ}₂ | \hat{σ} | P-value |
| (160, 160, 5.0) | 1000 | 160.10 | 159.86 | 4.985 | 160.10 | 159.86 | 4.985 | 0.449 |
| (180, 160, 5.0) | 1000 | 180.24 | 159.81 | 5.084 | 180.24 | 159.81 | 5.090 | 0.0 |
| | 10000 | 179.92 | 159.98 | 5.034 | 179.91 | 159.99 | 5.054 | 0.0 |
| | 100000 | 180.02 | 159.99 | 4.968 | 180.01 | 159.99 | 4.985 | 0.0 |
| (170, 160, 5.0) | 1000 | 170.31 | 159.70 | 5.165 | 170.31 | 159.71 | 5.167 | 2.0 × 10⁻¹⁵⁸ |
| | 10000 | 169.96 | 160.04 | 4.951 | 169.96 | 160.04 | 4.958 | 0.0 |
| | 100000 | 170.00 | 160.00 | 5.025 | 169.99 | 160.01 | 5.028 | 0.0 |
| (165, 160, 5.0) | 1000 | 164.83 | 159.56 | 4.932 | 164.83 | 159.57 | 4.932 | 2.6 × 10⁻⁵⁶ |
| | 10000 | 164.98 | 159.91 | 5.011 | 164.98 | 159.92 | 5.012 | 0.0 |
| | 100000 | 165.01 | 160.01 | 4.990 | 165.01 | 160.01 | 4.991 | 0.0 |

The estimation accuracy of the present algorithm can be evaluated by comparison with the true solution from the simulation data. The individuals are divided into a group having the haplotype CCTC and a group not having the haplotype CCTC according to the permutated diplotype configurations at the time of generation of the simulation data, means μ₁ and μ₂ thereof are obtained, and the overall standard deviation based on μ₁ and μ₂ is obtained, thus obtaining the values in the sample statistics section of Table 2. From comparison between statistics obtained from the samples and the results of estimation by the present algorithm, it is concluded that errors under the 4-locus simulation data condition setting are about 0.3% at the maximum, and that the estimation accuracy of the present algorithm is markedly high. The accuracy of estimation of the means of the phenotype associated with a particular haplotype is also high. It can be understood therefrom that it is virtually possible to detect a haplotype associated with a phenotype from data at least on the order of 1000 individuals.

The results of test calculation on simulation data using the 6-locus haplotype distribution performed to examine the estimation accuracy in the case of including loci at which linkage disequilibrium is weak are shown in the estimation/test result section of Table 3. The values shown in the calculation estimation/test result section were obtained under the conditions where μ₁=μ₂, σ=5 for the null hypothesis and μ₁≢μ₂=160, σ=5 for alternative hypothesis with respect to different sample sizes N.

TABLE 3
Results of estimation using 6-locus simulation data

| Conditions of | | | | | | | | |
|---|---|---|---|---|---|---|---|---|
| population | | Statistics of samples | | | Estimation/test results | | | |
| (μ₁, μ₂, σ) | N | μ₁ | μ₂ | σ | \hat{μ}₁ | \hat{μ}₂ | \hat{σ} | P-value |
| (160, 160, 5.0) | 1000 | 159.77 | 159.99 | 4.875 | 159.85 | 159.95 | 4.876 | 0.750 |
| (180, 160, 5.0) | 1000 | 179.81 | 159.99 | 4.923 | 179.54 | 160.17 | 5.333 | 0.0 |
| | 10000 | 180.19 | 160.03 | 5.022 | 179.66 | 160.34 | 5.742 | 0.0 |
| | 100000 | 180.03 | 159.98 | 5.000 | 179.52 | 160.30 | 5.722 | 0.0 |
| (170, 160, 5.0) | 1000 | 169.65 | 160.00 | 5.148 | 169.51 | 160.10 | 5.252 | 8.5 × 10⁻¹³⁰ |
| | 10000 | 169.88 | 160.09 | 5.023 | 169.62 | 160.24 | 5.202 | 0.0 |
| | 100000 | 169.98 | 160.00 | 4.990 | 169.72 | 160.16 | 5.179 | 0.0 |
| (165, 160, 5.0) | 1000 | 164.84 | 159.85 | 4.771 | 164.79 | 159.89 | 4.794 | 5.2 × 10⁻⁵¹ |
| | 10000 | 164.88 | 160.08 | 5.028 | 164.75 | 160.15 | 5.073 | 0.0 |
| | 100000 | 164.96 | 160.02 | 5.004 | 164.84 | 160.09 | 5.048 | 0.0 |

The same calculation as that in the case of the 4-locus data values is performed. That is, the individuals are divided into a group having the haplotype ACCGTC and a group not having the haplotype ACCGTC, and means μ₁ and μ₂ thereof are obtained, and the overall standard deviation based on μ₁ and μ₂ is obtained, as thus obtaining the values in the sample statistics section of Table 3. From comparison between statistics obtained from the samples and the results of estimation by the present algorithm, it is apparent that a phenotype associated with a particular haplotype can be separated under the 6-locus simulation data condition setting. However, if
|μ₁−μ₂|
is large, for example, in the case where μ₁=180 or μ₁=170, the maximum likelihood estimate
\hat{σ}
of the standard deviation tends to become larger. This is due to the characteristics of the standard deviation expected value calculation equation used in the present algorithm.
When
|μ₁−μ₂|
is large, the difference between
(ψᵢ−μ₁)²
and
(ψᵢ−μ₂)²
is large. For example, if μ₁ is a correct solution, the probability that
(ψᵢ−μ₂)²
is larger than
(ψᵢ−μ₁)²
is high. If no diplotype configuration is uniquely determined, the sum of the squares of the deviations distributed is larger in comparison with that in the case of the correct solution. In the 6-locus distribution in Table 1, haplotypes of a comparatively low frequency also appear because of inclusion of the loci at which linkage disequilibrium is weak. Therefore, no diplotype configuration is uniquely determined in some of the individuals, resulting in a reduction in estimation accuracy.

Owing to the method of calculating an expected value of a standard deviation in the present algorithm, the estimation accuracy is increased in a case where the difference between the means is small and in a case where the number of individuals in which a diplotype configuration is uniquely determined is high. Therefore, it is concluded that it is preferable to identify a haplotype block before application of the present algorithm. Also, from observation of the diplotype configuration distribution as a calculation result, it can be concluded that the estimation accuracy is reduced if the distribution is broad.

Accuracy of Estimation of Diplotype Configuration

Table 4 shows, from the results of 4-locus test calculation, diplotype distributions which are distributions of posterior probabilities of haplotype frequency distributions in the first ten of the individuals in the case where μ₁=165 or μ₂=160, σ=5.0 and N=1000

TABLE 4
Diplotype configuration distributions in first ten
individuals in 4-locus simulation data

| | | | | | Posterior |
|---|---|---|---|---|---|
| | | | | Posterior | probability |
| | QTL | | | probability | with QTL |
| | phenotype | Diplotype | | in haplotype | phenotype |
| No. | value | configuration | | estimation | weight |
| 1 | 157.1 | GCCT | GCCT | 1.0000 | 1.0000 |
| 2 | 170.3 | CCTC | CTCC | 0.9993 | 0.9999 |
| | | CCCC | CTTC | 0.0007 | 0.0001 |
| 3 | 173.4 | CCTC | GCCT | 1.0000 | 1.0000 |
| 4 | 158.2 | CTCT | GCCT | 1.0000 | 1.0000 |
| 5 | 170.6 | CTCT | GCCT | 1.0000 | 1.0000 |
| 6 | 162.4 | CTCC | CTCC | 1.0000 | 1.0000 |
| 7 | 161.6 | CTCC | GCCT | 0.9975 | 0.9975 |
| | | CTCT | GCCC | 0.0025 | 0.0025 |
| 8 | 149.4 | CTCC | CTCC | 1.0000 | 1.0000 |
| 9 | 164.3 | CCTC | GCCT | 1.0000 | 1.0000 |
| 10 | 165.1 | CTCC | GCCT | 0.9975 | 0.9975 |
| | | CTCT | GCCC | 0.0025 | 0.0025 |

The difference between the two inferences is small because the diplotype configurations are concentrated in the 4-locus data. In more detail, in comparison between the posterior probability obtained by using the haplotype frequency distribution estimated by the EM algorithm and the posterior probability obtained by considering the QTL phenotype, the value of the latter posterior probability calculated with respect to the individual 2 having the haplotype CCTC is higher.

Table 5 shows, from the results of 6-locus test calculation, diplotype distributions are distributions of posterior probabilities of haplotype frequency distributions in the ten duals from the fifty-first to the sixtieth under the same conditions μ₁=165 or μ₂=160, σ=5.0 and N=1000 as those in the above-described 4-locus test calculation.

TABLE 5
Diplotype configuration distributions of the first ten
individuals in 6-locus simulation data

| | | | | | Posterior |
|---|---|---|---|---|---|
| | | | | Posterior | probability |
| | QTL | | | probability | with QTL |
| | phenotype | Diplotype | | in haplotype | phenotype |
| No. | value | configuration | | estimation | weight |
| 51 | 157.3 | AGCGCT | AGCACT | 1.0000 | 1.0000 |
| 52 | 172.0 | AGCGCT | AGCGCT | 1.0000 | 1.0000 |
| 53 | 152.6 | ACTGCC | AGCGCT | 0.9993 | 0.9993 |
| | | ACTGCT | AGCGCC | 0.0007 | 0.0007 |
| 54 | 155.5 | AGCGCT | AGCGCT | 1.0000 | 1.0000 |
| 55 | 167.8 | ACCGTC | GCTGCT | 0.8871 | 0.9619 |
| | | ACTGCT | GCCGTC | 0.1129 | 0.0381 |
| 56 | 161.7 | ACTGCC | ACTGCC | 1.0000 | 1.0000 |
| 57 | 165.7 | ACTGCC | AGCGCT | 0.9993 | 0.9993 |
| | | ACTGCT | AGCGCC | 0.0007 | 0.0007 |
| 58 | 153.9 | ACTGCC | AGCGCT | 0.9993 | 0.9993 |
| | | ACTGCT | AGCGCC | 0.0007 | 0.0007 |
| 59 | 157.0 | ACTGCC | AGCGCT | 0.9993 | 0.9993 |
| | | ACTGCT | AGCGCC | 0.0007 | 0.0007 |
| 60 | 166.7 | ACTGCC | ACTGCC | 1.0000 | 1.0000 |

From comparison between the posterior probability obtained by using the haplotype frequency distribution estimated by the EM algorithm and the posterior probability obtained by considering the QTL phenotype, it can be understood that the value of the latter posterior probability calculated with respect to the individual 55 having the haplotype ACCGTC is higher.

The results of estimation of diplotype configuration distributions shown in Tables 4 and 5 show that the diplotype configuration estimation accuracy is increased if a haplotype associated with a phenotype is held, and that the present algorithm comprising a model of a particular genetic effect which is association between a phenotype and a haplotype is capable of increasing the accuracy of estimation of genetic information on individuals.

Empirical Distribution of Statistic −2log(L₀ₘₐₓ/Lₘₐₓ) Under Null Hypothesis

An empirical distribution of the statistic −²log(L₀ₘₐₓ/Lₘₐₓ) was examined by simulation under the null hypothesis. The null hypothesis corresponds to setting μ₁=μ₂. One value of the statistic −2log(L₀ₘₐₓ/Lₘₐₓ) is determined with respect to one sample. A multiplicity of samples are generated by simulation, and a distribution of the statistic is examined to obtain an empirical distribution.

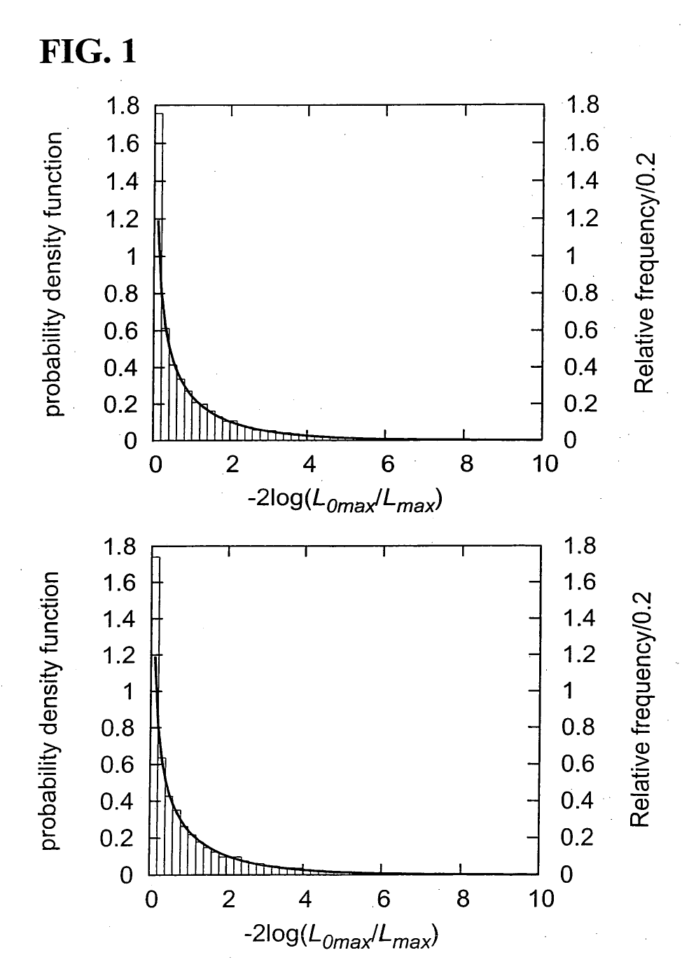

FIG. 1 comprises histograms relating to distributions of the test statistic −2log(L₀ₘₐₓ/Lₘₐₓ) obtained by simulation using a 4-locus haplotype distribution. FIG. 1 shows that the test statistic asymptotically follows x² distribution with I degree of freedom. N=100 and the number of samples=10000 in the left histogram in FIG. 1, while N=1000 and the number of samples=10000 in the right histogram in FIG. 1. In FIG. 1, the histograms are shown as bar graphs, while the probability density function corresponding to x² distribution with 1 degree of freedom is indicated by a curve.

Table 6 shows the estimated distribution parameters and the type I error rate. Simultions were performed in which samples having different sample sizes N=100 and N=1000 are repeatedly extracted under the conditions: μ₁μ₂=160 and σ=5 according to the null hypothesis. Sample extraction was performed 10000 times with respect to each set of parameters.

TABLE 6
Type I error in samples generated from a population according to null hypothesis

| | | | | | | | |
|---|---|---|---|---|---|---|---|
| | | | Number | | | | |
| | | | of | \hat{μ}₁ | \hat{μ}₂ | \hat{σ} | |
| μ₁ | μ₂ | N | samples | (mean ± SD) | (mean ± SD) | (mean ± SD) | type I error |

In the case of 4 loci

| | | | | | | | |
|---|---|---|---|---|---|---|---|
| 160 | 160 | 100 | 10000 | 160.01 ± 0.753 | 160.01 ± 0.676 | 4.936 ± 0.353 | 0.0496 |
| 160 | 160 | 1000 | 10000 | 160.00 ± 0.237 | 160.00 ± 0.213 | 4.995 ± 0.112 | 0.0514 |

In the case of 6 loci

| | | | | | | | |
|---|---|---|---|---|---|---|---|
| 160 | 160 | 100 | 10000 | 160.00 ± 0.822 | 159.99 ± 0.635 | 4.933 ± 0.352 | 0.0606 |
| 160 | 160 | 1000 | 10000 | 160.00 ± 0.256 | 160.00 ± 0.201 | 4.994 ± 0.110 | 0.0541 |

The type I error rate is 4.96% in the case where N=100 and the number of samples is 10000, and 5.14% in the case where N=1000 and the number of samples is 10000. Each error rate value is close to a level of significance of 5%. Table 6 also shows the estimated distribution parameters and the type I error rate obtained by simulation using s 6-locus haplotype distribution. The type I error rate is 6.06% in the case where N=100 and the number of samples is 10000, and 5.41% in the case where N=1000 and the number of samples is 10000. Each error rate value is close to the level of significance 5% but slightly higher. These results show that the type I error rate tends to also increase slightly in the test if the diplotype configurations are not concentrated.

Therefore, it is thought that in use of the present algorithm the accuracy is increased even in the test if the number of individuals in which a diplotype configuration is uniquely determined is large. Consequently, it is concluded that it is preferable to identify a haplotype block before application of the present algorithm.

Evaluation of Power of the Test

The power of the test was evaluated by performing a simulation under the alternative hypothesis. The type II error rate with respect to y, i.e., the power of the test, is evaluated by changing γ in μ₁=μ₂+γσ. A sample was generated with respect to each value of γ to evaluate the power of the test by simulation.

That is, the power of the test can be evaluated by changing γ in μ₁=μ₂+γσ and evaluating the distribution of the test statistic −2log(L₀ₘₐₓ/Lₘₐₓ). Table 7 shows the results of evaluation of the power of the test by changing γ=(μ₁-μ₂)/σ in a simulation using a 4-locus haplotype distribution.

TABLE 7
Power of test by simulation using 4-locus haplotype distribution

| | | | Number | | | | |
|---|---|---|---|---|---|---|---|
| | | | of | \hat{μ}₁ | \hat{μ}₂ | \hat{σ} | Power |
| μ₁ | μ₂ | N | samples | (mean ± SD) | (mean ± SD) | (mean ± SD) | of test |
| 160 | 160 | 100 | 10000 | 160.01 ± 0.753 | 160.01 ± 0.676 | 4.936 ± 0.353 | 0.0496 |
| 160.5 | 160 | 100 | 10000 | 160.50 ± 0.756 | 160.00 ± 0.683 | 4.937 ± 0.348 | 0.0848 |
| 161 | 160 | 100 | 10000 | 161.00 ± 0.751 | 160.01 ± 0.683 | 4.947 ± 0.355 | 0.1721 |
| 161.5 | 160 | 100 | 10000 | 161.50 ± 0.754 | 159.99 ± 0.675 | 4.939 ± 0.355 | 0.3255 |
| 162 | 160 | 100 | 10000 | 162.01 ± 0.753 | 159.99 ± 0.670 | 4.934 ± 0.354 | 0.5217 |
| 162.5 | 160 | 100 | 10000 | 162.49 ± 0.745 | 160.01 ± 0.686 | 4.933 ± 0.355 | 0.6875 |
| 163 | 160 | 100 | 10000 | 162.99 ± 0.747 | 159.99 ± 0.676 | 4.932 ± 0.356 | 0.8409 |
| 164 | 160 | 100 | 10000 | 164.00 ± 0.748 | 160.01 ± 0.690 | 4.940 ± 0.353 | 0.9750 |
| 165 | 160 | 100 | 10000 | 165.01 ± 0.758 | 160.01 ± 0.677 | 4.948 ± 0.356 | 0.9977 |
| 167 | 160 | 100 | 10000 | 166.99 ± 0.749 | 160.01 ± 0.678 | 4.937 ± 0.354 | 0.9988 |
| 160 | 160 | 1000 | 10000 | 160.00 ± 0.237 | 160.00 ± 0.213 | 4.995 ± 0.112 | 0.0514 |
| 160.3 | 160 | 1000 | 10000 | 160.30 ± 0.240 | 160.00 ± 0.213 | 4.993 ± 0.110 | 0.1567 |
| 160.6 | 160 | 1000 | 10000 | 160.60 ± 0.236 | 160.00 ± 0.213 | 4.994 ± 0.112 | 0.4688 |
| 161 | 160 | 1000 | 10000 | 161.00 ± 0.235 | 150.00 ± 0.214 | 4.997 ± 0.112 | 0.8837 |
| 161.3 | 160 | 1000 | 10000 | 161.31 ± 0.232 | 160.00 ± 0.215 | 4.993 ± 0.111 | 0.9849 |
| 161.6 | 160 | 1000 | 10000 | 161.60 ± 0.235 | 160.00 ± 0.209 | 4.995 ± 0.112 | 0.9991 |
| 162 | 160 | 1000 | 10000 | 162.00 ± 0.235 | 160.00 ± 0.212 | 4.996 ± 0.113 | 1.0000 |
| 165 | 160 | 1000 | 10000 | 164.99 ± 0.234 | 150.00 ± 0.214 | 4.996 ± 0.111 | 1.0000 |

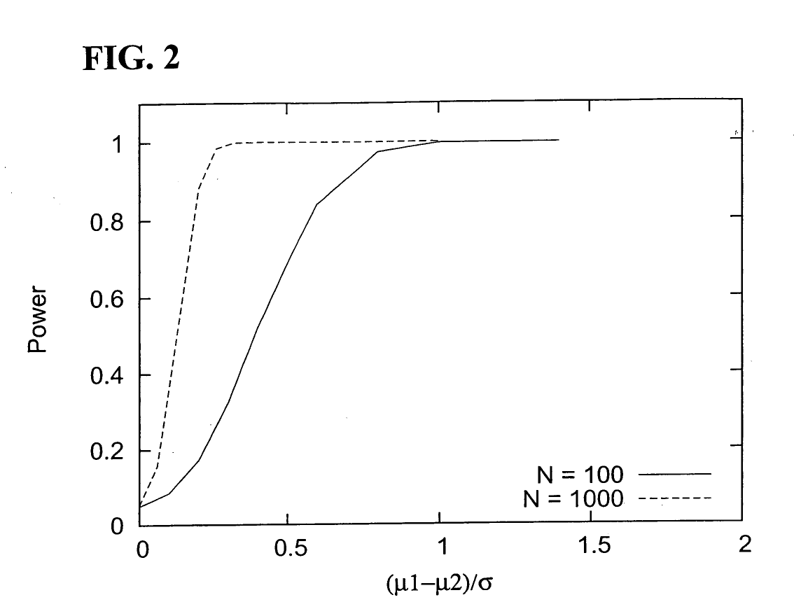

FIG. 2 also shows the power of the test with respect to γ=(μ₁−μ₂)/σ. When N=100 indicated by the solid line in FIG. 2), the power of the test is approximately equal to 1.0 if μ₁−μ₂=5, that is, γ=(μ₁−μ₂)/σ=1.0. When N=1000 (indicated by the broken line in FIG. 2), the power of the test is approximately equal to 1.0 if μ₁-μ₂=1.3, that is, γ=(μ₁−μ₂)/σ=0.3. Consequently, it is thought that the power of the test of the present algorithm is sufficiently high.

<Analysis of Real Data>

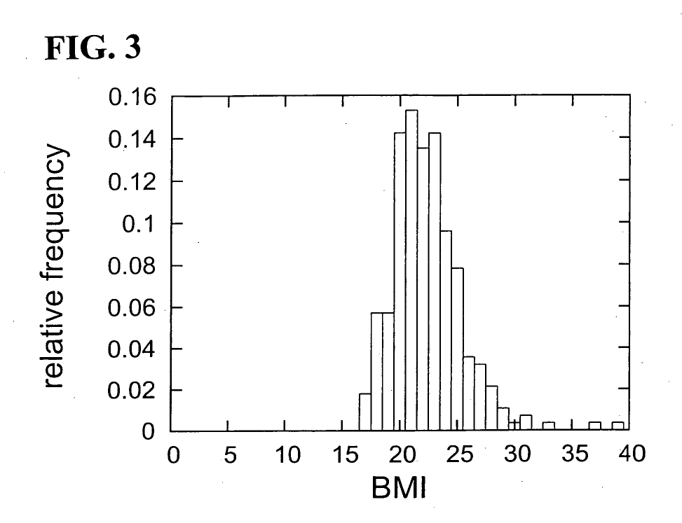

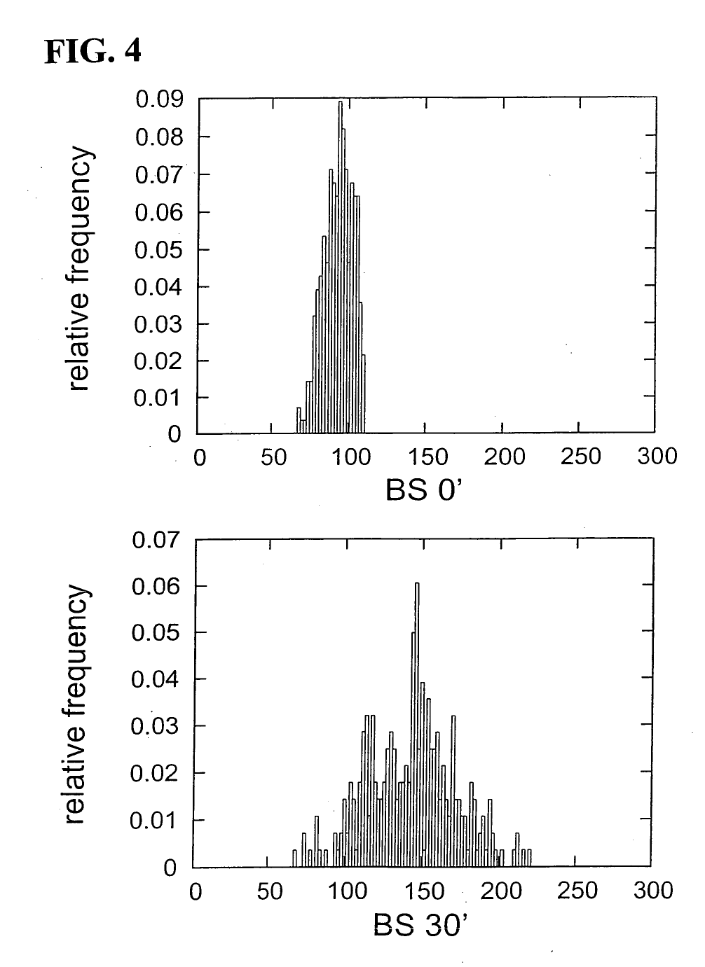

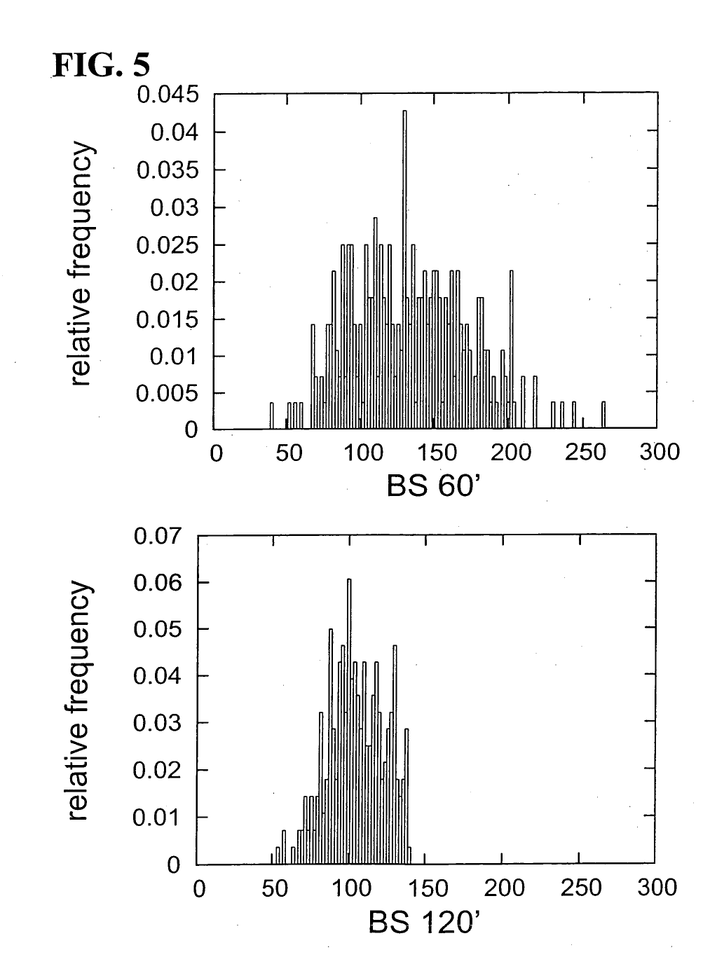

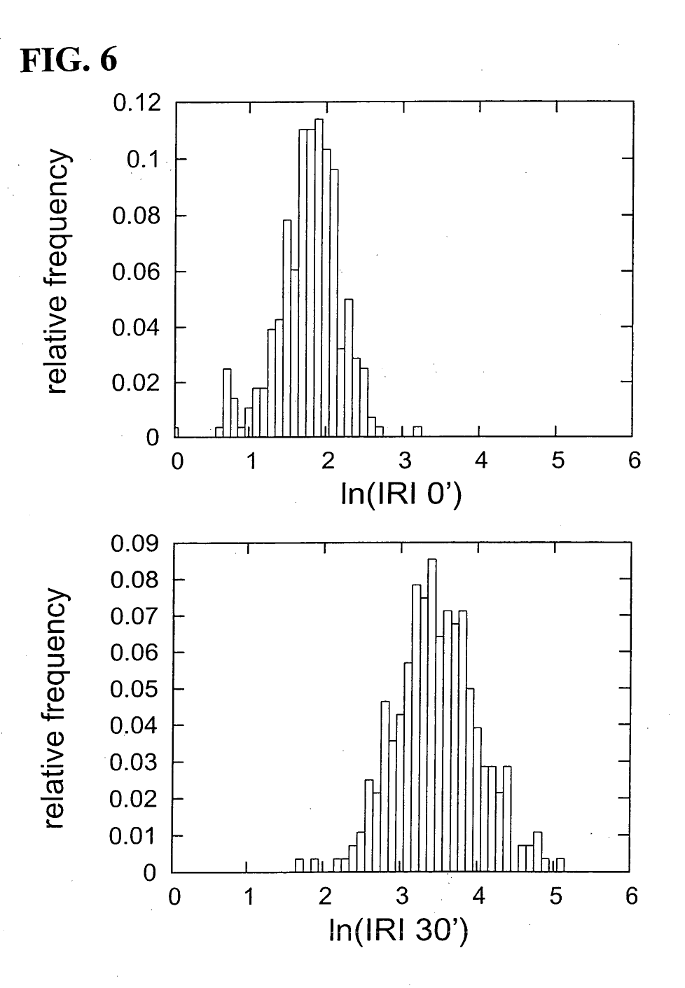

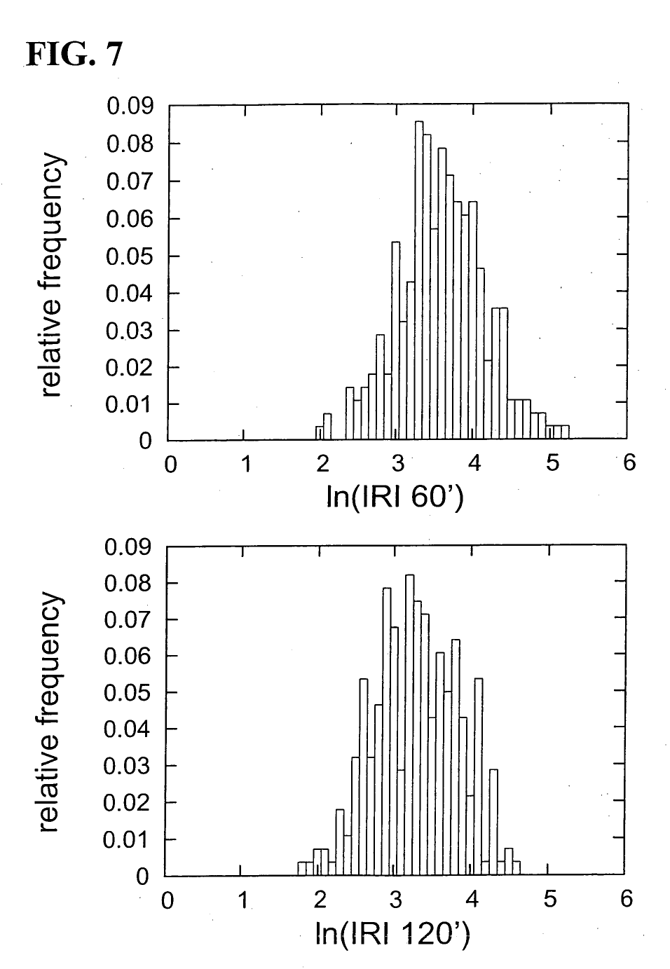

Analysis based on the present algorithm was performed by using 3SNPs genotype data and test data obtained in a study on the relation between the CAPN10 gene and diabetes (Horikawa Y, Oda N, Cox NJ, Li X, Orho-Melander M, Hara M, Hinokio Y et al. (2000) Genetic variation in the gene encoding calpain-10 is associated with type 2 diabetes mellitus. Nat Genet 26:163-175). The CAPNIO gene is a gene associated with the type II diabetes, and test data such as BMI and the blood sugar value relating to the CAPN10 gene have been obtained. FIG. 3 shows the distributions of the test data obtained by this study. Each distribution can be approximated with a normal distribution.

In the first stage of analysis, haplotype frequencies were obtained by using the haplotype estimation of the present algorithm. Table 8 shows SNPs frequencies and HWE test P-values, and Table 9 shows haplotype frequencies. Also, Table 10 shows criteria for linkage disequilibrium between each loci.

TABLE 8
SNPs frequencies of CAPN10 gene

| | Major | | Minor | | HWE test |
|---|---|---|---|---|---|
| Locus | allele | Frequency | allele | Frequency | P-value |
| 1 | 1 | 0.9448 | 2 | 0.0552 | 0.868 |
| 2 | 2 | 0.6263 | 1 | 0.3737 | 0.481 |
| 3 | 1 | 0.7242 | 2 | 0.2758 | 0.279 |

TABLE 9
Haplotype frequencies of CAPN10 gene

| | | Frequency when linkage |
|---|---|---|
| Haplotype | Frequency | disequilibrium is assumed to exist |
| 121 | 0.5696 | 0.4285 |
| 112 | 0.2588 | 0.0974 |
| 111 | 0.1078 | 0.2557 |
| 221 | 0.0468 | 0.0250 |
| 122 | 0.0087 | 0.1632 |
| 212 | 0.0070 | 0.0057 |
| 222 | 0.0012 | 0.0095 |

TABLE 10
Criteria for linkage disequilibrium of CAPN10 gene

| Locus 1 | Locus 2 | D | D′ | r² |
|---|---|---|---|---|
| 1 | 2 | −0.0138 | −0.6709 | 0.0157 |
| 1 | 3 | −0.0094 | −0.6148 | 0.0084 |
| 2 | 3 | 0.1628 | 0.9424 | 0.5669 |

Next, haplotypes 121, 112, 111, 221, 122, 122 and 212 were assumed to exist as haplotypes associated with a phenotype from the results of haplotype estimation, and analysis using the present algorithm was performed. The analysis results shown in Table 11 show that haplotype 112 is significant with BS 30′ and BS 60′, and that haplotype 122 is significant with BS 0′. The estimation results shown in FIG. 12 show that
\hat{μ}₁>\hat{μ}₂
with respect to the combination of BS 30′ and haplotype 112 and the combination of BS 60′ and haplotype 112, and haplotype 112 significantly heighten BS 30′ and BS 60′. On the other hand, the estimation result with respect to BS 0′ and haplotype 122 shows
\hat{μ}₁<\hat{μ}₂

TABLE 11
Results of test on CAPN10 gene by QTLhaplo (P-value)

| | Haplotype^{a} | | | | | |
|---|---|---|---|---|---|---|
| | 111 | 112 | 121 | 122 | 212 | 221 |
| BMI | 0.6945 | 0.8070 | 0.8212 | 0.2023 | 0.6404 | 0.6388 |
| BS 0′ | 0.1359 | 0.9367 | 0.3346 | 0.0202 | 0.3308 | 0.7343 |
| BS 120′ | 0.1629 | 0.3311 | 0.7492 | 0.8296 | 0.7076 | 0.3930 |
| BS 30′ | 0.3446 | 0.0140 | 0.6959 | 0.9199 | 0.9823 | 0.2765 |
| BS 60′ | 0.5855 | 0.0406 | 0.3630 | 0.4207 | 0.6450 | 0.6953 |
| IRI 0′ | 0.8445 | 0.8333 | 0.6737 | 0.3340 | 0.4997 | 0.6336 |
| IR.T 120′ | 0.5277 | 0.5698 | 0.2823 | 0.3505 | 0.9530 | 0.7354 |
| IRI 30′ | 0.8457 | 0.4698 | 0.5068 | 0.2656 | 0.8750 | 0.7758 |
| IRI 60′ | 0.8581 | 0.0589 | 0.3135 | 0.3548 | 0.8576 | 0.7383 |

TABLE 12
Results of estimation on CAPN10 gene by qtlhaplo

| | Haplotype^{a} | | | | | | | | |
|---|---|---|---|---|---|---|---|---|---|
| | 111 | | | 112 | | | 121 | | |
| | \hat{μ}₁ | \hat{μ}₂ | \hat{σ} | \hat{μ}₁ | \hat{μ}₂ | \hat{σ} | \hat{μ}₁ | \hat{μ}₂ | \hat{σ} |
| BMI | 22.4 | 22.3 | 3.01 | 22.4 | 22.26 | 3.01 | 22.3 | 22.4 | 3.01 |
| BS 0′ | 91.0 | 93.2 | 9.35 | 92.8 | 92.74 | 9.39 | 93.0 | 91.7 | 9.37 |
| BS 30′ | 139.2 | 143.3 | 28.5 | 147.1 | 138.9 | 28.2 | 142.8 | 141.2 | 28.5 |
| BS 60′ | 130.5 | 133.8 | 39.0 | 138.5 | 129.0 | 38.8 | 134.2 | 128.9 | 39.0 |
| BS 120′ | 102.1 | 105.9 | 18.2 | 106.4 | 104.2 | 18.2 | 105.3 | 104.5 | 18.2 |
| IRI 0′ | 1.78 | 1.77 | 0.423 | 1.77 | 1.78 | 0.424 | 1.78 | 1.75 | 0.424 |
| IRI 30′ | 3.49 | 3.48 | 0.540 | 3.51 | 3.46 | 0.539 | 3.47 | 3.52 | 0.539 |
| IRI 60′ | 3.58 | 3.60 | 0.562 | 3.67 | 3.54 | 0.559 | 3.61 | 3.53 | 0.561 |
| IRI 120′ | 3.25 | 3.30 | 0.545 | 3.31 | 3.27 | 0.545 | 3.31 | 3.22 | 0.544 |
| | Haplotype | | | | | | | | |
| | 122 | | | 212 | | | 221 | | |
| | \hat{μ}₁ | \hat{μ}₂ | \hat{σ} | \hat{μ}₁ | \hat{μ}₂ | \hat{σ} | \hat{μ}₁ | \hat{μ}₂ | \hat{σ} |
| BMI | 20.3 | 22.3 | 3.00 | 21.6 | 22.3 | 3.01 | 22.6 | 22.3 | 3.01 |
| BS 0′ | 82.8 | 92.9 | 9.30 | 88.6 | 92.8 | 9.37 | 92.2 | 92.8 | 9.38 |
| BS 30′ | 141.2 | 142.5 | 28.5 | 141.9 | 142.5 | 28.5 | 136.8 | 143.1 | 28.5 |
| BS 60′ | 118.7 | 133.3 | 39.0 | 125.1 | 133.3 | 39.0 | 130.3 | 133.4 | 39.0 |
| BS 120′ | 103.4 | 105.2 | 18.2 | 102.0 | 105.2 | 18.2 | 102.3 | 105.5 | 18.2 |
| IRI 0′ | 1.58 | 1.78 | 0.423 | 1.91 | 1.77 | 0.423 | 1.74 | 1.78 | 0.424 |
| IRI 30′ | 3.76 | 3.48 | 0.539 | 3.52 | 3.48 | 0.540 | 3.51 | 3.48 | 0.540 |
| IRI 60′ | 3.35 | 3.60 | 0.561 | 3.64 | 3.60 | 0.562 | 3.56 | 3.60 | 0.562 |
| IRI 120′ | 3.05 | 3.29 | 0.545 | 3.27 | 3.29 | 0.545 | 3.26 | 3.29 | 0.545 |

The fact that the number of corresponding samples is small due to the low frequency of haplotype 122 from the first is thought to be the cause of making the test result significant.

The above-described results of analysis using real data show that the present algorithm is capable of maximum likelihood estimation of haplotype frequencies, a posterior probability distribution of diplotype configurations in individuals and QTL phenotype parameters by using genotype data and continuous-value phenotype data. Therefore, the present algorithm enables a test on the association between a QTL phenotype and a haplotype on the individual level on the basis of genotype data and continuous-value phenotype data. Also, the present -algorithm enables maximum likelihood estimation of a range in which a quantitative phenotype is taken depending on condition that the quantitative phenotype can be taken in different ranges depending on the existence/nonexistence of a particular haplotype. Further, the present algorithm is capable of obtaining a maximum likelihood estimate of a relative risk from such ranges.

On the other hand, analysis using the 3SNPs genotype data and test data obtained by the study on the relationship between the CPN10 gene and diabetes was carried out with respect to incomplete haplotype H_{I} set as an object to be tested, as mentioned above. Through this data, it has already been shown that the haplotype 112 is significant with BS 30′ and BS 60′, which are test data (see Tables 8 through 12).

TABLE 13

| Incomplete | | | | |
|---|---|---|---|---|
| haplotype | P-value | μ₁ | μ₂ | σ |
| 121 | 6.96E−01 | 142.8 | 141.2 | 28.5 |
| 112 | 1.40E−02 | 147.1 | 138.9 | 28.2 |
| 111 | 3.45E−01 | 139.2 | 143.3 | 28.5 |
| 221 | 2.77E−01 | 136.8 | 143.1 | 28.5 |
| 1** | 6.35E−01 | 142.6 | 129.0 | 28.5 |
| *1* | 1.16E−01 | 144.7 | 139.3 | 28.4 |
| *2* | 8.92E−01 | 142.4 | 143.1 | 28.5 |
| **1 | 3.92E−01 | 142.0 | 147.2 | 28.5 |
| 1*1 | 7.51E−01 | 142.3 | 144.0 | 28.5 |

As shown in Table 13, haplotype “122” exhibits the lowest P-value in the analysis results, and is significant. However, when htSNP for constructing the incomplete haplotype is determined, haplotype “122” having a low frequency is excluded from the object to be tested. Thus, an incomplete haplotype is used to enable a search for “a haplotype associated with a phenotype”.

Another example of an application using incomplete haplotype H_{I} is analysis using an incomplete haplotype as an object to be tested, which was performed on genotype data including four SNPs relating to the CHST3 gene and data on a cumulative dose of methotrexate to a point in time at which GPT, which is an index of liver functions, exceeded 45, with respect to ninety-eight cases of articular rheumatism who developed symptoms in which GPT exceeded 45. Table 14 shows the results of this analysis.

TABLE 14

| Incomplete | | | | |
|---|---|---|---|---|
| haplotype | P-value | μ₁ | μ₂ | σ |
| ACTC | 3.80E−01 | 811.3 | 577.5 | 677.5 |
| AATC | 2.24E−01 | 756.8 | 463.4 | 670.3 |
| TACT | 4.11E−01 | 567.5 | 770.3 | 678.4 |
| TATT | 8.92E−02 | 715.0 | 107.1 | 656.1 |
| *CTC | 4.87E−01 | 495.3 | 688.0 | 680.4 |
| *ATC | 2.24E−01 | 756.8 | 463.4 | 670.3 |
| *ATT | 8.91E−02 | 715.0 | 107.1 | 656.1 |
| *ACT | 4.11E−01 | 567.5 | 770.3 | 678.4 |
| *A** | 5.82E−01 | 757.0 | 604.2 | 682.3 |
| **T* | 6.04E−01 | 836.7 | 621.7 | 682.6 |
| ***T | 8.22E−01 | 618.3 | 672.4 | 684.9 |
| ***C | 7.55E−01 | 553.2 | 657.0 | 684.4 |
| *AT* | 3.82E−02 | 873.5 | 394.5 | 642.2 |

Haplotype “*AT*” exhibits the lowest P-value in the analysis results, which shows that haplotype “*AT*” is more significant than haplotype “AATC” or “TATT” using information on all the loci, and that the incomplete haplotype “AT” on the second and third loci is associated more strongly with the cause of the phenotype. That is, it can be understood that the cause locus is associated with each of haplotypes “AATC” and “TATT”, and that masking of the first and fourth loci causes the association with the phenotype to be strongly exhibited. This result shows that a phenotype associated with a plurality of haplotypes can be detected if an incomplete haplotype is used.

According to the present invention, it is possible to perform maximum likelihood estimation of haplotype frequencies in a population, a distribution of posterior probabilities of diplotype configurations of individuals and a means and a standard deviation determining a distribution of a quantitative phenotype by using genotype data and phenotype data taking a continuous value, with no need to determine the diplotype configurations of the individuals without question. The algorithm in accordance with the present invention is used to enable, for example, a test on the association between the existence of a haplotype and the range of a value indicated by a quantitative phenotype using genotype data and continuous-value phenotype data obtained from a cohort study or a clinical trial.

1. A method of estimating a means and a standard deviation determining a distribution of a quantitative phenotype, said method comprising:
a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration including a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration including the predetermined haplotype and the phenotype data distribution taking a continuous value; and
a step b of obtaining the means and the standard deviation determining a distribution of a quantitative phenotype from the maximum likelihood estimates obtained in said step a.

2. The method of estimating a means and a standard deviation determining a distribution of a quantitative phenotype according to claim 1, wherein, in said step a, the maximum likelihood (Lₘₐₓ) is obtained by maximizing the following expression (I):
$$\begin{matrix}L ( Θ , \vec{μ} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , \vec{μ} , σ ) \\ over Θ , \\ \vec{μ} ( which is \vec{μ} = ( {μ}_{1} , {μ}_{2} , … , {μ}_{{L}^{2}} ) \end{matrix} \qquad ( I )$$
and which is a means of distributions of all possible diplotype configurations) and σ (standard deviation), and the maximum likelihood (L₀ₘₐₓ) is obtained by maximizing the following expression (II):
$$\begin{matrix}L ( Θ , \vec{\overline{μ}} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , \vec{\overline{μ}} , σ ) \\ over Θ , \\ \vec{\overline{μ}} ( which is \vec{\overline{μ}} = ( {μ}_{1} , {μ}_{1} , … , {μ}_{1} ) \end{matrix} \qquad ( II )$$
and which is a means constant with respect to all possible diplotype configurations) and σ (standard deviation) (wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢ=ωᵢ|dᵢ=aₖ,\vec{μ}, σ)
in the above expression (I) denotes a probability density function for development of a quantitative phenotype x under
di∈D₊
where D₊ is a set of diplotype configurations including an element in a set of haplotypes related to the predetermined phenotype, and dᵢ is a diplotype configuration of the ith individual in N individuals;
$$f ( {ψ}_{i} = {ω}_{i} \vert {d}_{i} = {a}_{k} , \vec{\overline{μ}} , σ )$$
in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

3. The method of estimating a means and a standard deviation determining a distribution of a quantitative phenotype according to claim 1, wherein genotype data and phenotype data observed in a predetermined population and obtained as a result of a cohort study or a clinical trial are used.

4. The method of estimating a means and a standard deviation determining a distribution of a quantitative phenotype according to claim 1, wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.

5. A program for estimating a means and a standard deviation determining a distribution of a quantitative phenotype, said program enabling a computer to execute:
a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration including a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration having the predetermined haplotype and the phenotype data distribution taking a continuous value; and
a step b of obtaining the means and the standard deviation determining a distribution of a quantitative phenotype from the maximum likelihood estimates obtained in said step a.

6. The program of estimating a means and a standard deviation determining a distribution of a quantitative phenotype according to claim 5, wherein, in said step a, the maximum likelihood (Lₘₐₓ) is obtained by maximizing the following expression (I):
$$\begin{matrix}L ( Θ , \vec{\overline{μ}} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , \vec{\overline{μ}} , σ ) \\ over Θ , \\ \vec{μ} ( which is \vec{μ} = ( {μ}_{1} , {μ}_{2} , … , {μ}_{{L}^{2}} ) \end{matrix} \qquad ( I )$$
and which is a means of distributions of all possible diplotype configurations) and σ (standard deviation), and the maximum likelihood (L₀ₘₐₓ) is obtained by maximizing the following expression (II):
$$L ( Θ , {μ}^{\vec{\_}} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , {μ}^{\vec{\_}} , σ ) \\ over Θ , {μ}^{\vec{\_}} ( which is \\ {μ}^{\vec{\_}} = ( {μ}_{1} , {μ}_{1} , … , {μ}_{1} ) \qquad ( II )$$
and which is a means constant with respect to all possible diplotype configurations) and σ (standard deviation)
(wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢ=ωᵢ|dᵢ=aₖ, \vec{μ}, σ)
in the above expression (I) denotes a probability density function for development of a quantitative phenotype x under
di∈D₊
where D₊ is a set of diplotype configurations including an element in a set of haplotypes related to the predetermined phenotype, and dᵢ is a diplotype configuration of the ith individual in N individuals;
$$f ( {ψ}_{i} = {ω}_{i} \vert {d}_{i} = {a}_{k} , {μ}^{\vec{\_}} , σ )$$
in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

7. The program of estimating a means and a standard deviation determining a distribution of a quantitative phenotype according to claim 5, wherein genotype data and phenotype data observed in a predetermined population and obtained as a result of a cohort study or a clinical trial are used.

8. The program of estimating a means and a standard deviation determining a distribution of a quantitative phenotype according to claim 5, wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.

9. A method of testing the association between a haplotype and a quantitative phenotype, said method comprising:
a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration having a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration including the predetermined haplotype and the phenotype data distribution taking a continuous value; and
a step b of obtaining a likelihood ratio from the maximum likelihood (L₀ₘₐₓ) and the maximum likelihood (Lₘₐₓ) obtained in said step a, and testing, with respect to x² distribution, the hypothesis that there is an association between the diplotype configuration including the predetermined haplotype and the predetermined quantitative phenotype.

10. The method of testing the association according to claim 9, wherein, in said step a, the maximum likelihood (Lₘₐₓ) is obtained by maximizing the following expression (I):
$$L ( Θ , \vec{μ} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , {μ}^{\vec{\_}} , σ ) \\ over Θ , \vec{μ} ( which is \\ \vec{μ} = ( {μ}_{1} , {μ}_{2} , … , {μ}_{{L}^{2}} ) \qquad ( I )$$
and which is a means of distributions of all possible diplotype configurations) and σ (standard deviation), and the maximum likelihood (L₀ₘₐₓ) is obtained by maximizing the following expression (II):
$$L ( Θ , {μ}^{\vec{\_}} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , {μ}^{\vec{\_}} , σ ) \\ over Θ , {μ}^{\vec{\_}} ( which is \\ {μ}^{\vec{\_}} = ( {μ}_{1} , {μ}_{1} , … , {μ}_{1} ) \qquad ( II )$$
and which is a means constant with respect to all possible diplotype configurations) and σ (standard deviation)
(wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢ=ωᵢ|dᵢ=aₖ, \vec{μ}, σ)
in the above expression (I) denotes a probability density function for development of a quantitative phenotype x under
di∈D₊
where D₊ is a set of diplotype configurations including an element in a set of haplotypes related to the predetermined phenotype, and dᵢ is a diplotype configuration of the ith individual in N individuals;
$$f ( {ψ}_{i} = {ω}_{i} \vert {d}_{i} = {a}_{k} , {μ}^{\vec{\_}} , σ )$$
in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

11. The method of testing the association according to claim 9, wherein, in said step b, −2log(Lₘₐₓ/L₀ₘₐₓ) (where “log” denotes natural logarithm) is obtained as a statistic, and wherein, in a case where there is no association between the diplotype configuration including the predetermined haplotype and the quantitative phenotype, and where the statistic therefore follows asymptotically x² distribution with 1 degree of freedom, it is determined that no association can be said to exist between the diplotype configuration including the predetermined haplotype and the predetermined quantitative phenotype, when the statistic does not exceed the limit value x² (which is a value in x² distribution with 1 degree of freedom at which a cumulative distribution function becomes 1-α (where α is a level of significance of the test)), and it is determined that there is an association between the predetermined haplotype and the predetermined quantitative phenotype, when the statistic exceeds the limit value x².

12. The method of testing the association according to claim 9, wherein genotype data and phenotype data taking a continuous value observed in a predetermined population and obtained as a result of a cohort study or a clinical trial are used.

13. The method of testing the association according to claim 9, wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.

14. A program for testing the association between a haplotype and a quantitative phenotype, said program enabling a computer to execute:
a step a of calculating the maximum likelihood (L₀ₘₐₓ) on the basis of genotype data and phenotype data taking a continuous value by using as parameters haplotype frequencies and a means and a standard deviation determining a distribution of a quantitative phenotype, under the hypothesis that there is no association between a diplotype configuration having a predetermined haplotype and a phenotype data distribution taking a continuous value, and maximum likelihood estimates and the maximum likelihood (Lₘₐₓ) of haplotype frequencies and the means and the standard deviation determining a distribution of the quantitative phenotype obtained by maximizing the likelihood under the hypothesis that there is an association between the diplotype configuration including the predetermined haplotype and the phenotype data distribution taking a continuous value; and
a step b of obtaining a likelihood ratio from the maximum likelihood (L₀ₘₐₓ) and the maximum likelihood (Lₘₐₓ) obtained in said step a, and testing, with respect to x² distribution, the hypothesis that there is an association between the predetermined haplotype and the predetermined quantitative phenotype.

15. The program of testing the association according to claim 14, wherein, in said step a, the maximum likelihood (Lₘₐₓ) is obtained by maximizing the following expression (I):
$$L ( Θ , \vec{μ} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , {μ}^{\vec{\_}} , σ ) \\ over Θ , \vec{μ} ( which is \\ \vec{μ} = ( {μ}_{1} , {μ}_{2} , … , {μ}_{{L}^{2}} ) \qquad ( I )$$
and which is a means of distributions of all possible diplotype configurations) and σ (standard deviation), and the maximum likelihood (L₀ₘₐₓ) is obtained by maximizing the following expression (II):
$$L ( Θ , {μ}^{\vec{\_}} , σ ) ∝ {\prod }_{i = 1}^{N} {\sum }_{{a}_{k} ∈ {A}_{i}} P ( {d}_{i} = {a}_{k} \vert Θ ) f ( {ψ}_{i} = {ω}_{i} \vert {d}_{i} = {a}_{k} , {μ}^{\vec{\_}} , σ ) \\ over Θ , {μ}^{\vec{\_}} ( which is \\ {μ}^{\vec{\_}} = ( {μ}_{1} , {μ}_{1} , … , {μ}_{1} ) \qquad ( II )$$
and which is a means constant with respect to all possible diplotype configurations) and σ (standard deviation)
(wherein Θ in the above expressions (I) and (II) denotes the vector of the haplotype frequencies;
P(dᵢ=aₖ|Θ)
in the above expressions (I) and (II) denotes the probability that the ith individual has a value aₖ realizing a diplotype configuration, dᵢ being a random variable representing a diplotype configuration of the ith individual;
ƒ(ψᵢ=ωᵢ|dᵢ=aₖ, \vec{μ}, σ)
in the above expression (I) denotes a probability density function for development of a quantitative phenotype x under
di∈D₊
where D₊ is a set of diplotype configurations including an element in a set of haplotypes related to the predetermined phenotype, and dᵢ is a diplotype configuration of the ith individual in N individuals;
$$f ( {ψ}_{i} = {ω}_{i} \vert {d}_{i} = {a}_{k} , {μ}^{\vec{\_}} , σ )$$
in the above expression (II) denotes the probability that the quantitative phenotype x is exhibited under
di∉D₊
; Ψᵢ denotes a phenotype as a probability variable for the ith individual; ωᵢ denotes a phenotype as an actually measured value of the ith individual; aₖ denotes a possible diplotype of the kth individual; and Aᵢ denotes a set of diplotypes matching genotype data gᵢ on the ith individual).

16. The program of testing the association according to claim 14, wherein, in said step b, −2log(Lₘₐₓ/L₀ₘₐₓ) (where “log” denotes natural logarithm) is obtained as a statistic, and wherein, in a case where there is no association between the diplotype configuration including the predetermined haplotype and the quantitative phenotype, and where the statistic therefore follows asymptotically x² distribution with I degree of freedom, it is determined that no association can be said to exist between the diplotype configuration including the predetermined haplotype and the predetermined quantitative phenotype, when the statistic does not exceed the limit value x² (which is a value in x² distribution with 1 degree of freedom at which a cumulative distribution function becomes 1-α (where α is a level of significance of the test)), and it is determined that there is an association between the predetermined haplotype and the predetermined quantitative phenotype, when the statistic exceeds the limit value x².

17. The program of testing the association according to claim 14, wherein genotype data and phenotype data taking a continuous value observed in a predetermined population and obtained as a result of a cohort study or a clinical trial are used.

18. The program of testing the association according to claim 14, wherein loci for giving information for discrimination between individual haplotypes and loci having redundant information depending on a combination of the loci having the discrimination information are identified on the basis of the haplotype frequencies used as parameters, and the identified loci are masked to restrictively define a set of haplotypes to be tested.
